(19) United States
(12) Patent Application Publication
(10) Pub. No.: US 2022/0049219 A1
(43) Pub. Date: Feb. 17, 2022

(54) AUTOMATED GENERATION AND ANALYSIS OF ORGANOIDS

(71) Applicant: Max-Planck-Gesellschaft zur Förderung der Wissenschaften e.V., Munchen (DE)

(72) Inventors: Jan Markus BRUDER, Munster (DE); Henrik RENNER, Munster (DE); Hans Robert SCHÖLER, Munster (DE); Martha Anna GRABOS, Munster (DE); Mandy OTTO, Munster (DE)

(21) Appl. No.: 17/275,084

(22) PCT Filed: Sep. 11, 2019
(86) PCT No.: PCT/EP2019/074197

§ 371 Date: Mar. 10, 2021

(30) Foreign Application Priority Data
Sep. 11, 2018 (EP) 18193698.0

(51) Int. Cl. C12N 5/0797; G01N 1/30; G01N 33/50

(52) U.S. Cl. CPC C12N 5/0623; C12N 2506/45; G01N 33/5082; G01N 1/30

(57) ABSTRACT

The present invention relates to a method of producing organoids, said method comprising or consisting of: (a) seeding a plurality of tissue-specific precursor cells into a container; (b) allowing to occur (i) aggregation of said cells; and (ii) maturation of the aggregate formed in (i) into a single organoid; wherein said method does not comprise embedding of said cells or said aggregates into a gel.

The present invention relates to a method of producing organoids, said method comprising or consisting of: (a) seeding a plurality of tissue-specific precursor cells into a container; (b) allowing to occur (i) aggregation of said cells; and (ii) maturation of the aggregate formed in (i) into a single organoid; wherein said method does not comprise embedding of said cells or said aggregates into a gel.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Three dimensional (3D) cell culture in the form of organ-like micro tissues (“organoids”) has found a rapid following over the past few years. The potential of organoids to mimic cellular niches more closely than 2D cell cultures promises to develop next generation high throughput screens (HTS) that can provide more relevant predictions of drug efficacy and toxicity. These may allow better modeling of uniquely human diseases such as Parkinson's, Alzheimer's and other disorders with complex interactions of several cell types in specific cellular niches.

However, while the current state of the art allows the generation of many types of organoids, the rigorous standardized organoid production and quantification methods needed for screening have been elusive. Most established protocols yield complex yet very heterogeneous organoids without predictable morphology, cellular composition, and local cell organization. Obligatory extensive manual handling including cumbersome matrix embedding steps render most protocols challenging for industrial scale up. Furthermore, existing analysis methods either do not scale well (e.g. sectioning and immunostaining, RNA sequencing) or rely mostly on measurements of size and morphology (Hou, Y., Konen, J., Brat, D. J., Marcus, A. I. & Cooper, L.A.D. TASI: A software tool for spatial temporal quantification of tumor spheroid dynamics. Sci Rep 8, 7248 (2018); and Kang, A., Seo, H. I., Chung, B. G. & Lee, S. H. Concave microwell array-mediated three dimensional tumor model for screening anticancer drug-loaded nanoparticles. Nanomedicine 11, 1153-1161 (2015)) or overall cell viability (Vlachogiannis, G. et al. Patient-derived organoids model treatment response of metastatic gastrointestinal cancers. Science 359, 920-926 (2018) but do not provide information with cellular resolution. Finally, data revealing cell-cell interactions in the context of a three-dimensional niche are scarce.

In view of the limitations of the prior art, the technical problem underlying the present invention can be seen in the provision of improved means and methods for generating, handling and/or analyzing organoids.

This technical problem is solved by the subject-matter of the claims.

Accordingly, in a first aspect, the present invention relates to a method of producing organoids, said method comprising or consisting of: (a) seeding a plurality of tissue-specific precursor cells into a container; (b) allowing to occur (i) aggregation of said cells; and (ii) maturation of the aggregate formed in (i) into a single organoid; wherein said method does not comprise embedding of said cells or said aggregates into a gel.

The term “organoid” has its art-established meaning. Accordingly, it relates to a miniaturized version of an organ that is produced in vitro in three dimensions. An organoid shows microanatomy and cellular function resembling that of native tissues in vivo. Typically, an organoid comprises multiple organ-specific cell types, wherein said cell types are spatially organized in a defined manner, in the case of neural organoids typically in layers. Generally, said defined spatial organisation of multiple cell types is a result of self-organization occurring during the formation of the organoid. Organoids comprise distinct cell types that interact spatially and/or functionally with each other, preferably in a self-organized matrix. The term “self-organized matrix” refers to the spatial arrangement of cells with different cellular function and identity such that they resemble in part or entirely the cellular arrangement found in native tissues in vivo. In addition, they can be maintained for extended period of times in culture. “Extended periods” in this context are typically more than about 100 or more than about 200 days.

In terms of function, an organoid is generally capable of recapitulating one or more specific functions of the corresponding organ.

Therefore, organoids are distinct from spheroids and aggregates. Spheroids are cellular aggregates that are typically of smaller size (about 10 to about 200 μm diameter) than organoids and lack distinct cellular organization, such as distinct layers. In accordance with the invention, “spheroid” and “aggregate” refer to the same subject-matter. Preferably, organoids, in terms of size, are at least 2 times, at least 5 times, more preferably at least 10 times larger than spheroids. A preferred measure of size is the diameter of the largest cross-section.

As regards production of organoids, there is a common understanding in the art that cells or aggregates thereof have to be cultured in a three-dimensional (3D) medium. A hallmark of such 3D media is that they are solid or semisolid, typically gel-like and/or comprised of a natural or artificial hydrogel. The use of 3D media or gels is viewed as being indispensable for the culture of organoids. The terms “3D medium” and “gel” are used interchangeably in this disclosure. Said gel may be selected from a basement membrane-like matrix, matrigel, collagen, dextran and extracellular matrix. Such materials are well-known in the art and described, for example, in Semin Cancer Biol. 2005 October; 15(5):378-86, Matrigel: basement membrane matrix with biological activity; Kleinman HK1, Martin GR; and Gjorevski, N. et al. Designer matrices for intestinal stem cell and organoid culture. Nature 539, 560-564 (2016).

The present inventors surprisingly found that, contrary to common understanding, the mentioned 3D medium or gel is dispensable. Dispensing with a 3D medium or gel entails several advantages. First, handling of the material during culture is less cumbersome and more amenable to automation. In fact, the high degree of automation of the procedures in accordance with the present invention is unprecedented. Secondly, the avoidance of gel is understood to render the produced organoids more homogenous. As will be described in more detail below, the aspect of homogeneity includes size, but is not limited thereto. Organoids in accordance with the present invention have preferably a size between about 500 μm and about 2 mm and a standard deviation of less than about 20% from the mean.

The step of seeding cells in accordance with (a), followed by step (b), provides formation of a single organoid in said container. As will be described in more detail below, and noting that the methods of the present invention are amenable to automation as well as high throughput, a plurality of containers such as wells of a microwell plate can be handled in an automated manner and simultaneously, thereby enabling a format of the methods of the present invention wherein a plurality of containers are handled, wherein each container, as a result of steps (a) and (b), contains a single organoid.

Otherwise, step (a) of seeding cells as well as step (b) may follow art-established procedures. Preferred embodiments thereof in accordance with the present invention are described further below.

Another important feature is the use of tissue-specific precursor cells. While art-established methods in many instances use embryonic stem cells or pluripotent cells, the use of tissue-specific precursor cells is a means of ensuring and/or increasing homogeneity of the obtained organoids.

The step of seeding may be implemented, for example, by adding a defined number of cells to a medium allowing aggregation to occur. Suitable media are known in the art and described in more detail in the Examples. Preferred numbers of cells to be seeded into a given container are detailed further below.

None of the stages occurring during performance of the method of the invention is embedded into a 3D medium or gel. In particular, aggregates are not embedded into gel. This is distinct from conventional protocols providing for embedding of the aggregates into gel, typically after a period of 10 to 15 days. Contrary to the established opinion, the present inventors demonstrated that embedding into a gel is dispensable and provides for distinct advantages. These advantages include the possibility to perform production and handling of organoids in an automated manner which is amenable to scale up and high throughput.

In a preferred embodiment, (i) said organoids are neural organoids, preferably midbrain organoids or non-patterned homogeneous brain organoids; and said tissue-specific precursor cells are neuronal tissue-specific precursor cells, preferably small molecule neuronal precursor cells (smNPCs); (ii) said organoids have a reproducible or homogeneous size and/or cellular composition, homogenous preferably meaning a standard deviation of less than 20% of the mean or less; (iii) step (b) comprises (b-i) culturing in aggregation medium, preferably for about two days, said aggregation medium preferably comprising polyvinyl alcohol; (b-ii) culturing in a maturation medium; and (b-iii) preferably, between (b-i) and (b-ii), culturing in ventral patterning medium, preferably for about four days; (iv) said plurality of cells is between about 100 and about 1000000, preferably about 10000 cells; and/or (v) said container is a well of a multiwell plate, wherein preferably a plurality of wells or each well of said multiwell plate is seeded with a plurality of said cells, such that a multiwell plate is obtained, wherein a plurality of wells or each well contains one single organoid.

As such, in preferred embodiment (i) which relates to producing neural organoids, preference is given to a specific type of neuronal precursor cells, namely small molecule neuronal precursor cells (smNPCs). These specific neuronal precursor cells (see, e.g. Reinhardt, P. et al. Derivation and expansion using only small molecules of human neural progenitors for neurodegenerative disease modeling. PLoS One 8, e59252 (2013)) have the advantage that, in terms of factors required in the medium, small molecules are sufficient. In particular, protein-based growth factors are not necessary. The term “small molecule” has its art-established meaning. As such, it relates a low molecular weight organic compound, typically with a molecular weight below 1000 Da, preferably below 900 Da. Biological macromolecules such as nucleic acids, proteins, polypeptides and polysaccharides are not to be subsumed under the term “small molecule”.

Said midbrain organoids, owing to their convenient and automated generation in accordance with the invention, are also referred to as “automated midbrain organoids (AMOs)”.

As stated above, a second preferred embodiment of neural in accordance with the invention are non-patterned homogeneous brain organoids (NABOs). When preparing NABOs, differentiation is performed without patterning factors. As a consequence, NABOs are not directed towards a specific fate or brain region like the midbrain. Instead, they are general neuronal organoids or general brain-like organoids. Preferably, after 28 days of culture, NABOs contain early postmitotic and mature neurons as characterized by the markers DCX, MAP2, and Tubb3, as well as synapses (Synapsin). Over time, they mature further to include glial cells including astrocytes as evidenced by GFAP and S100 expression. Overall, they possess a high degree of structural homogeneity both in size and internal organization. With the exception of the absence of patterning factors during maturation, the preparation of NABOs parallels that of AMOs.

The term “medium” as used in the above definition of smNPCs refers to the medium used in the two-dimensional culture of said smNPCs. To explain further, said two-dimensional culture serves to generate sufficient amounts of smNPCs by means of cell division. Accordingly, said two-dimensional culture typically precedes the methods in accordance with the first aspect. In other words, while method of the first aspect may comprise the mentioned two-dimensional culture as a step preceding step (a), it does not have to.

Having said that, it is an important feature of smNPCs, when used for the method in accordance with the first aspect of the present invention, that step (a) of said method of the first aspect also does not require proteins or peptides as factors, but only small molecules. This is also apparent from the list of constituents of the aggregation medium given further below. Preferred small molecules are Smoothened Agonist (SAG) and CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile).

Certain preferred midbrain organoids in accordance with the present invention comprise dopaminergic neurons. They mimic inter alia the behavior of the substantia nigra in the midbrain. In order to generate organoids with such properties, the herein disclosed step of ventral patterning is preferred.

The art-established step of embedding in a gel is a cumbersome process requiring manual intervention. Furthermore, and noting that the exact placement of an aggregate within a gel drop is difficult to reproduce, each aggregate, after having been placed within a drop of gel, typically experiences a different environment. These limitations and undesirable variations are overcome by the method in accordance with the first aspect. As a direct consequence thereof, and in accordance with item (ii) of the above-disclosed preferred embodiment, organoids obtained by the method of the invention are highly reproducible with regard to the properties. Such homogeneity refers to both within-batch variation as well as batch-to-batch variation. As regards the former, a typical standard deviation from the mean is less than 5%, and as regards the latter, a typical standard deviation from the mean is less than 25%, less than 20% or less than 15%. Preferred parameters used for determining the degree of homogeneity are size including diameter and/or cellular composition. The experimental data enclosed herewith provide evidence of said homogeneity. Improved homogeneity goes along with improved reproducibility and improved predictability.

A further feature of the method of the first aspect is that agitation, as commonly performed in art-established organoid culture in bioreactors, is dispensable. Accordingly, in a preferred embodiment, said method does not comprise stirring.

In addition to dispensing with the use of gel, the present inventors performed a further optimization of the conditions for aggregation and maturation. More specifically, and in accordance with item (iii) of the above-disclosed embodiment, the culturing in aggregation medium (a preferred composition of which will be described further below) is performed for about two days. This is a time span which is short in comparison to art-established protocols which art-established protocols provide for leaving the cells for extended period of times in the same medium which herein is referred to as “aggregation medium”. Culturing in aggregation medium is followed by culturing in maturation medium, wherein an optional intervening step which is preferable in the context of the production of midbrain organoids, provides for culturing in ventral patterning medium. The preferred early switch from aggregation medium to maturation medium or optionally to ventral patterning medium is an additional means of increasing homogeneity of the produced organoids.

In a further preferred embodiment, fetal calf serum (FCS) is not used for aggregation. In order to boost aggregation, it is preferred to add polyvinylalcohol (PVA), preferably in a concentration between 0.1% (w/v) and 1% (w/v).

Preferred numbers of cells in the plurality of cells to be seeded in accordance with item (a) of the method of the first aspect are given in item (v). An exemplary number of cells constituting said plurality of cells are about 9000 cells.

In line with the preferred automated and/or high throughput implementations, item (vi) provides for uses of multiwell plates. Importantly, preferably each well of a multiwell plate contains one single organoid.

This is advantageous in that batch effects such as effects due to paracrine signaling will not occur. To the extent paracrine signaling would be desired, the workflow can easily be changed such that more than one organoid is placed in a given container or well.

A particularly preferred implementation of the protocol for a generation of organoids in accordance with the invention is given in the corresponding subsection of Example 1.

In a second aspect, the present invention relates to an organoid or a plurality of organoids obtained by the method of any one of the preceding claims.

Owing to the distinct method of producing organoids, the obtained organoids are distinct from organoids of the prior art.

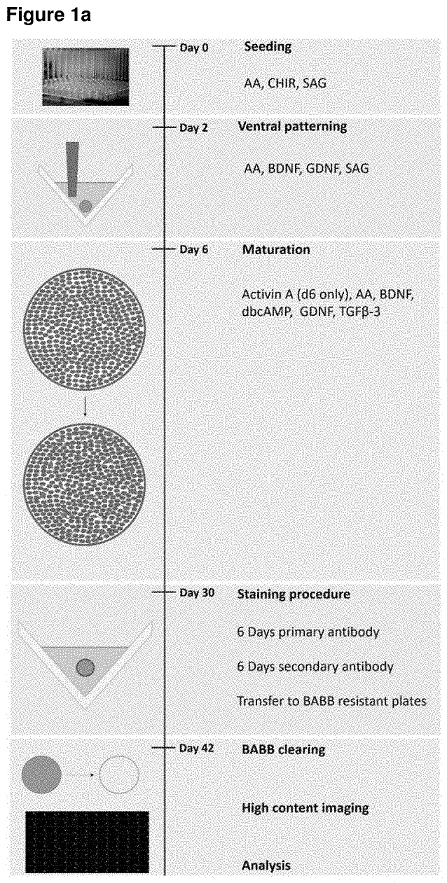

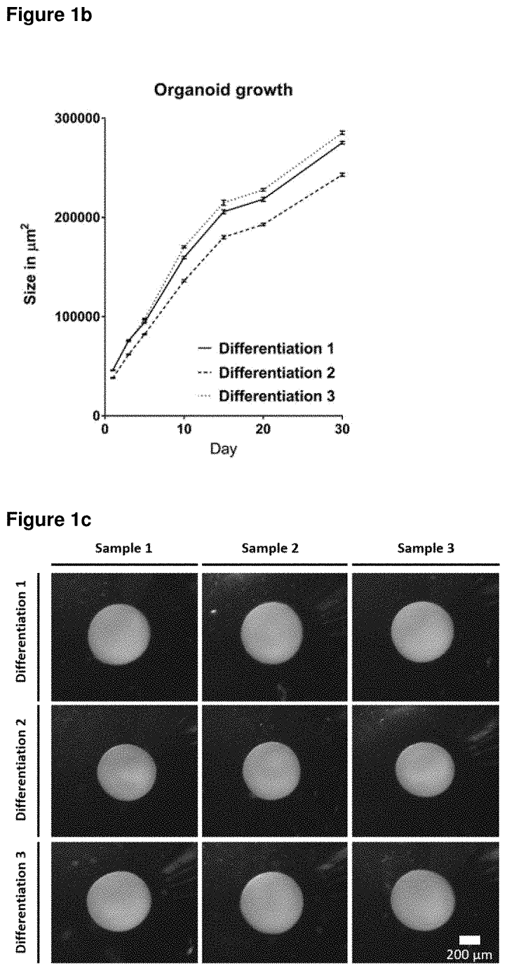

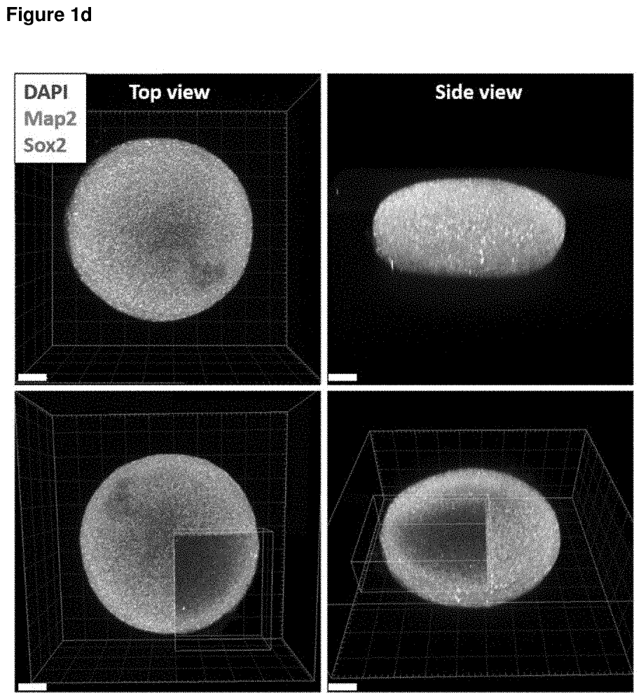

This includes a striking degree of homogeneity; see the section entitled “Homogeneity” of Example 2 as well as FIGS. 1 and 8 to 11. These figures include microscopic images which highlight the morphological heterogeneity as well as heterogeneity in size of a plurality of prior art or organoids as compared to the organoids of the present invention which are characterized by well-defined spherical shape and a very narrow distribution of geometric parameters such as the radius.

Accordingly, it is apparent that organoids in accordance with the present invention are inherently different from any organoids of the prior art, in particular in view of the unprecedented homogeneity across a population of organoids. Said homogeneity allows for uses which are not possible with prior art organoids, such uses including drug screening and toxicology screening (see also Examples).

Parameters for quantifying homogeneity of size and/or shape include the sphericity ψ which is defined as follows:

$$Ψ = \frac{{{π}^{\frac{1}{3}} ( 6 {V}_{p} )}^{\frac{2}{3}}}{{A}_{p}}$$

where Vₚ is volume and the particle and Aₚ is the surface area of the particle. Any particle which is not a sphere will have sphericity less than 1.

In a preferred embodiment, organoids in accordance with the invention have a sphericity between 0.85 and 1.0, more preferably between 0.9 and 1.0, and yet more preferably between 0.95 and 1.0.

To the extent homogeneity is to be determined on the basis of two-dimensional images, a two-dimensional counterpart of the above defined sphericity may be used. To give an example, roundness may be employed which is the ratio between the inscribed and the circumscribed circles, i.e. a maximum and a minimum size for circles that are just sufficient to fit inside and to enclose the shape under consideration, respectively. Accordingly, roundness of a two-dimensional object may be defined as R=rᵢ/r_{c}, wherein rᵢ and r_{c} are the radii of the inscribed and the circumscribed circles, respectively.

Preferred roundness values are between 0.85 and 1.0, more preferably between 0.9 and 1.0 and yet more preferably between 0.95 and 1.0.

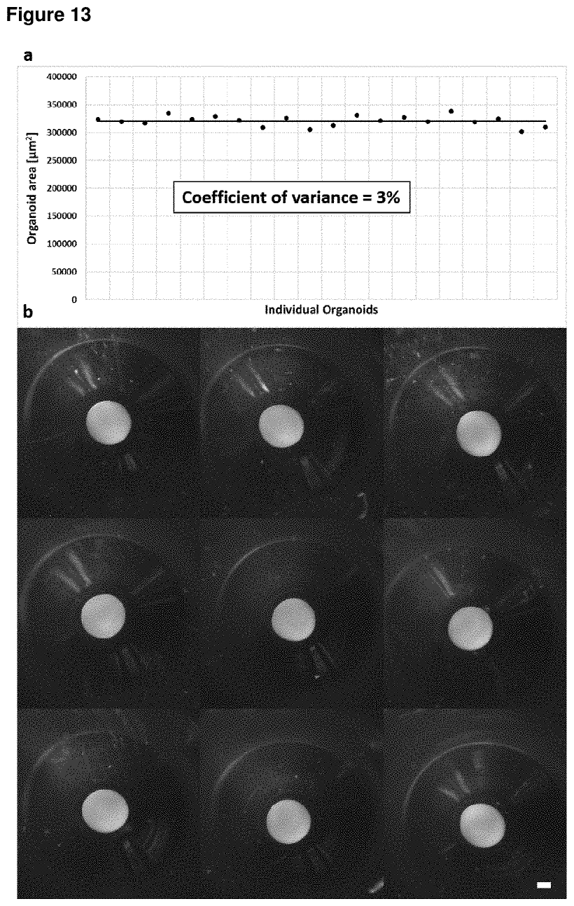

Further suitable parameters are the breadth of the distribution of the largest diameter d, the coefficient of variation (CV) of the largest diameter, the breadth of the distribution of the largest cross-section Aₘₐₓ, and the CV of said largest cross-section. Exemplary data for the CV of Aₘₐₓ for NABOs of the invention are given in FIG. 13.

As regards the breadth of the distribution of the maximum diameter d, it is preferred that 90% of the measured maximum diameters in a plurality of organoids obtained by the present invention is within +/−20% of the mean of the maximum diameter, more preferably within +/−10%. The same applies mutatis mutandis to the distribution of Aₘₐₓ.

Preferred ranges of CV for both d and Aₘₐₓ are less than 10%, less than 5%, less than 4% or about 3%.

Importantly, such homogeneity is achieved without resorting to any type of device capable of imposing a particular shape and/or homogeneity such as a mold. To explain further, it is conceivable to make use of a mold, for example a mold generated by a 3D printer, during producing organoids and/or thereafter. Such mold can impose a shape and/or homogeneity on organoids, wherein said organoids may be organoids which would not exhibit the shape and/or homogeneity imposed by the device, e.g. mold, in the absence of the device, e.g. mold. As a consequence, it is understood that in a preferred embodiment of the method of the first aspect, no use is made of a mold. Similarly, organoids in accordance with the invention are preferably organoids which have not been shaped by a mold, be it during producing or thereafter. The absence of said device or mold is a preferred embodiment of all aspects of the invention.

It is understood that the container in accordance with the first aspect does not act as mold or shaping device. Preferably, its dimensions are larger than the size of the organoids, such as at least 2-fold, at least 5-fold, at least 10-fold or at least 100-fold. “Dimension” may be the aperture of said container and/or its depth.

In a preferred embodiment, (a) said organoid(s) is/are (a) neural organoid(s), preferably (a) midbrain organoid(s) or (a) non-patterned homogeneous brain organoid(s); (b) said organoid(s) exhibit(s) (i) a plurality of concentric zones, each zone differing from any of the other zones with regard to cellular composition and organization, preferably at least three zones; and/or (ii) said organoid(s) exhibit tissue-specific cellular activity, preferably, in case of neural organoids, electrical activity in neurons; and/or (c) said plurality of organoids is homogenous in terms of structure and/or size; wherein said organoid or said plurality is preferably obtained by the method in accordance with the first aspect.

Related thereto, the invention provides, in a second aspect, an organoid or a plurality of organoids, wherein (a) said organoid(s) is/are (a) neural organoids, preferably (a) midbrain organoid(s) or (a) non-patterned homogeneous brain organoid(s); (b) said organoid(s) exhibit(s) (i) a plurality of concentric zones, each zone differing from any of the other zones with regard to cellular composition and organization, preferably at least three zones; and/or (ii) said organoid(s) exhibit tissue-specific cellular activity, preferably, in case of neural organoids, electrical activity in neurons.

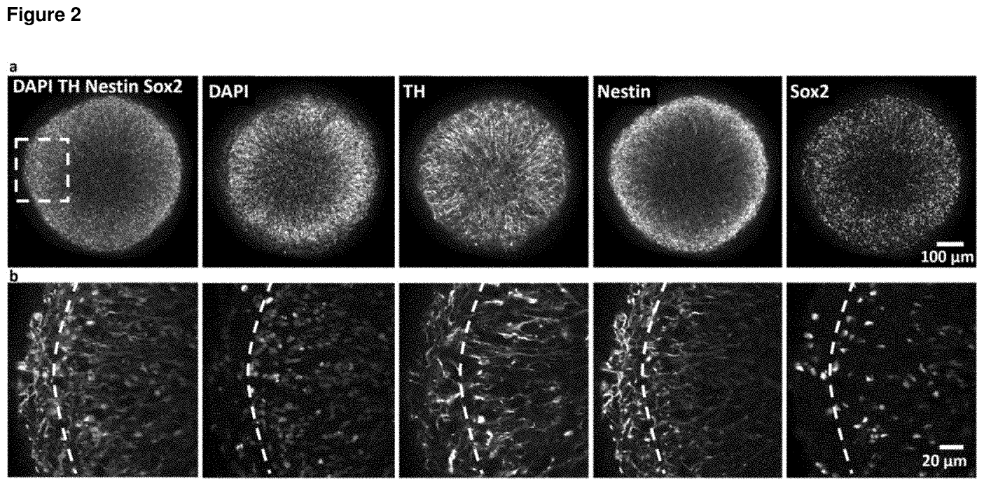

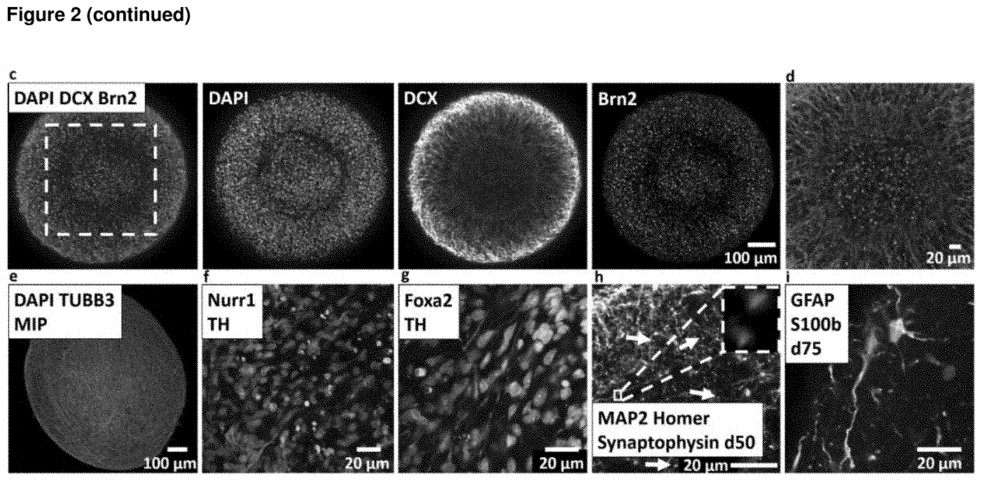

The mentioned concentric zones are further detailed in the Examples. Particularly preferred is the presence of four concentric zones. This can be seen in FIGS. 2a and 2c. A preferred cellular composition of the mentioned four concentric zones is described for midbrain organoids in accordance with the present invention in the subsection entitled “Automated midbrain organoids express typical neural and midbrain markers and show structural organization” of Example 2. Related to the above, organoids produced in accordance with the present invention are preferably spherical or exhibit radial symmetry.

Item (ii) provides for the above-mentioned recapitulation of organ-like behavior. Surprisingly, and as described in more detail in the subsection entitled “Calcium imaging reveals spontaneous and synchronized activity throughout entire organoids” of Example 2, organoids in accordance with the second and third aspect of the present invention are functionally connected across the entire organoid. In the context of the preferred neural organoids, the tissue-specific cellular activity observed across the entire organoid is an electrical activity, more specifically electrical activity involving a plurality of neurons, preferably in a synchronized manner. It is also preferred that organoids in accordance with the present invention produce and optionally secrete tissue-specific proteins.

In terms of monitoring tissue-specific proteins, an antibody directed against SOX2 may be used to detect changes in the amount of neural precursor cells present within the organoid. An antibody directed against MAP2 may be used to detect changes in the amount of more mature neural cells within the organoid.

In a fourth aspect, the present invention provides a multiwell plate, wherein a plurality of wells contain each one single organoid or each well contains one single organoid, wherein preferably a plurality of the organoids or each organoid is as defined in accordance with the second or third aspect and/or obtained by the method in accordance with the first aspect.

As noted above, preferred implementations of the methods of the invention involve the use of a plurality of containers, typically the wells of a multiwell plate. When performing the methods of the invention in such a format, the multiwell plate in accordance with the fourth aspect is a result of performing such method.

Related to the first aspect, the present invention provides, in a fifth aspect, the use of tissue-specific precursor cells for organoid production, wherein no use is made of a gel for embedding cells or aggregates, wherein preferably said tissue-specific precursor cells are neuronal tissue-specific precursor cells, preferably small molecule neuronal precursor cells (smNPCs).

As explained in the background section herein above, present workflows relating to organoids suffer from several deficiencies. One of these deficiencies is the art-established embedding into gel. This is addressed by the aspects of the invention disclosed above. A further deficiency of the prior art procedures is a consequence of the size of organoids and their opaque appearance in a microscope. As a consequence thereof, conventional staining or labeling approaches, while rendering an analysis of the surface of an organoid with penetration depths of about 50 to 100 μm possible, fail to enable a comprehensive analysis of all cells comprised in an organoid.

The present invention addresses this difficulty by providing, in a sixth aspect, a method of preparing organoids or spheroids for analysis, said method comprising or consisting of: (a) staining said organoids or spheroids; (b) performing tissue clearing with said organoids or spheroids.

Steps (a) and (b) of this method of the sixth aspect may be performed in any order. Having said that, preference is given to performing step (a) before step (b). This applies in particular in conjunction with the preferred embodiment of benzyl alcohol and benzyl benzoate (BABB)-based clearing described in more detail below.

Particularly preferred implementations of staining and tissue clearing are given in the corresponding subsections of Example 1.

In a preferred embodiment, (a) said staining is effected with (i) an antibody, preferably with a primary and with a secondary antibody, wherein staining with said primary antibody and/or said secondary antibody is effected for about 5 to about 10 days, preferably about 6 days; (ii) a fluorescent label; (iii) a luminescent label; (iv) a radioactive label; and/or (b) said clearing is (BABB)-based clearing wherein preferably said clearing is performed in cylco-olefin containers, more preferably in cylco-olefin multiwell plates.

As described in the examples, for the purpose of staining with antibodies, use is made of a solution which comprises Triton-X 100, preferably in a concentration between 0.1% (w/v) to 1.0% (w/v), more preferably 0.5% (w/v). This amounts to a specific adaptation to the staining of organoids developed by the present inventors.

The preferred duration of the staining procedure, i.e. about 5 to about 10 days, preferably about 6 days is a further specific adaptation to the staining of organoids.

BABB-clearing is described in, for example, Dent, J. A., Polson, A. G. & Klymkowsky, M. W. A whole-mount immunocytochemical analysis of the expression of the intermediate filament protein vimentin in Xenopus. Development 105, 61-74 (1989).

Other clearing methods, while being less preferred, may also be employed. Examples are X-clarity (Logos biosystems (2015): X-CLARITY™ Tissue Clearing System, User Manual, LBSM-0005 Ver 1.8, 205.05.28, www.logosbio.com), CUBIC (Susaki, E. A. et al. Advanced CUBIC protocols for whole-brain and whole-body clearing and imaging. Nat Protoc 10, 1709-1727 (2015), ScaleSQ (Hama, H. et al. ScaleS: an optical clearing palette for biological imaging. Nat Neurosci 18, 1518-1529 (2015) and Clear^{T} (Kuwajima, T. et al. ClearT: a detergent- and solvent-free clearing method for neuronal and non-neuronal tissue. Development 140, 1364-1368 (2013)).

Suitable labels include phalloidin, nuclear counter stains such as DAPI, luciferases and live/dead stains, i.e. dyes that indicate whether a cell is dead or alive. The latter are preferred in conjunction with toxicity screening described below.

Owing to the combination of staining and clearing, a sectioning of said organoids in order to make all cells amenable to analysis is no longer necessary. In other words, in a preferred embodiment (a) said method does not comprise sectioning of said organoids or spheroids and/or said staining is whole mount staining; and/or (b) said organoids are organoids of the second or third aspect or are obtained by the method of the first aspect.

Related to the method of the sixth aspect, the present invention, in a seventh aspect, provides a method of analysing organoids or spheroids, said method comprising or consisting of the method of the sixth aspect; and (c) analysis of stained and cleared organoids or spheroids, preferably (c-i) optical analysis, said optical analysis preferably comprising microscopy and/or image analysis; (c-ii) genetic analysis such as RNA sequencing; and/or (c-iii) protein analysis such as mass spectrometry or Western blotting. Thanks to the step clearing, all cells of the organoids can be analyzed, preferably rendered visible, and preferably at a single cell resolution level.

In view of the above disclosed aspects of the invention, in particular the method of producing organoids in accordance with the first aspect and the method of analyzing organoids in accordance with the seventh aspect, both of which are amenable to automation and high throughput, the present invention renders an automated and integrated method of preparation and analysis of organoids possible. This is subject of the eighth aspect which relates to a method of preparing and analysing organoids, said method comprising or consisting of the method of the first aspect and the method of the seventh aspect.

As explained in the background section, organoids are increasingly being recognized as valuable tools in the field of medicine including screening for lead compounds, toxicity testing, disease models and personalized medicine. Accordingly, in a ninth aspect, the present invention provides a method of identifying modulators of organoids, of organoid formation and/or of organoid-specific function, said method comprising or consisting of (a)(i) adding a test compound to an organoid, preferably of the second or third aspect or obtained by the method of the first aspect; (ii) adding a test compound to tissue-specific precursor cells, followed by performing the method of the first aspect; or (iii) performing the method of the first aspect, wherein a test compound is added at one or more time points during said performing the method of the first aspect; (b) performing the method of the seventh aspect; (c) comparing the result of said analysis in the presence of said test compound with the result of said analysis in the absence of said test compound, wherein a difference is indicative of a modulator.

In a preferred embodiment, (a) if said analysis is indicative of a functional improvement of said organoid, of organoid formation and/or of organoid-specific function, said test compound is a lead compound, said method optionally further comprising or further consisting of developing said lead compound to yield a drug; or (b) if said analysis is indicative of a decrease of function of said organoid and/or of negative interference with organoid formation and/or with organoid-specific function, this is indicative of said test compound being toxic.

The term “lead compound” as used herein refers to a compound which optionally may be subjected to optimization in order eventually become a drug. In the alternative, the lead compound as such may be a drug. The mentioned optimization may include an optimization of stability, pharmacokinetics and pharmacodynamics. Furthermore, the lead compound may be associated with a specific molecular target, i.e. it may be a binder preferably inhibitor of a target molecule, preferably a molecule occurring in cells comprised in the organoid which has been recognized as being disease-associated.

Possibly, but not necessarily related thereto, the mentioned aspect of toxicity testing may also be relevant in the context of drug development. In other words, it is conceivable that there is the concomitant presence of two readouts, one being indicative of a lead compound being useful drug candidate, and another one, possibly at a higher dose, being indicative of toxicity. Under such circumstances, i.e. beneficial effects at low concentrations and toxic effects at high concentration, the lead compound or drug would be equipped with a therapeutic window.

In a further aspect, the present invention provides the use of one or more organoids produced with the method of the first aspect or as defined in accordance with the second or third aspect as disease model. Diseases to be modeled may be genetic diseases. Also, disease may be induced by addition of pathogens and/or disease-inducing compounds.

In a preferred embodiment of all methods of the invention, said method is performed (a) in an automated manner; and/or (b) in high-throughput format, preferably using multiwell plates, a pipetting robot, automated liquid handling, a plate reader and/or means for plate transportation.

Art-established multiwell plates may be used, for example plates with 96, 384 or 1536 wells.

In a particular preferred embodiment, commercially available high throughput equipment may be directly used when performing methods of the present invention, i.e. no hardware adjustments are necessary.

In a tenth aspect, the present invention provides a kit comprising or consisting of (a) tissue-specific precursor cells, preferably neuronal tissue-specific precursor cells, more preferably smNPCs; (b) media, said media comprising or consisting of (b-i) aggregation medium, said aggregation medium preferably comprising polyvinyl alcohol; (b-ii) maturation medium; and (b-iii) optionally, ventral patterning medium.

Aggregation medium preferably consists of DMEM-F12 and Neurobasal Medium at a 1:1 ratio, enriched with 1:400 diluted N2 supplement, and 1:200 diluted B27 supplement without vitamin A, 1% penicillin/streptomycin/glutamine, 200 μM ascorbic acid, and the small molecules SAG (0.5 μM) and CHIR 99021 (3 μM).

A preferred ventral patterning medium is as follows: Same as aggregation medium, except that CHIR is removed and 0.5 ng/mL brain derived neurotrophic factor (BDNF) and 1 ng/mL glial cell line-derived neurotrophic factor (GDNF) are added.

A preferred maturation medium is as follows: Same as ventral patterning medium, except that SAG is removed and 0.5-1 ng/mL transforming growth factor beta 3 (TGFβ-3) and 100 μM dibutyryl cyclic adenosine monophosphate (dbcAMP) are added.

As regards the embodiments characterized in this specification, in particular in the claims, it is intended that each embodiment mentioned in a dependent claim is combined with each embodiment of each claim (independent or dependent) said dependent claim depends from.

For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The Figures show:

FIG. 1: Automation enables high throughput compatible production and analysis of homogenous midbrain organoids.
a Schematic overview of the automated HTS-compatible workflow used in this study including organoid generation and optical analysis. b Measurement of organoid size (area of the largest cross section) reveals low variation and parallel growth kinetics for 3 independent differentiations. Error bars represent standard error of the mean (SEM), n≥20). c Light microscopy images illustrating the morphological homogeneity of AMOs. Scale bar: 200 μm. d 3D rendering of confocal slices showing an entire organoid (d 25) stained for the neural marker Map2 and neural precursor marker Sox2 after whole-mount staining and clearing. Lower row: cut out to visualize internal structure. Scale bar 100 μm.

FIG. 2: Automated midbrain organoids express typical neural and midbrain markers and show signs of structural organization.
a Expression of the dopaminergic midbrain marker TH as well as the precursor markers Nestin and Sox2 is evenly distributed throughout the entire organoid at day 25, as shown by single confocal microscopy slices. The dotted box indicates the area shown in b. Here, higher magnification of the peripheral organoid region reveals two different zones with few nuclei but dense, circumferentially oriented neurites distally from the core and radial organization of TH positive neurons more proximally. c The expression patterns of DCX and Brn2 further illustrate the organization of neurons (DCX) and neural precursors (Brn2) in the core of AMOs into concentric zones. d Enlargement (of the dotted box in c highlighting the circumferential organization of neurons (DCX) surrounding the core. e Maximum intensity projection of fluorescent confocal images showing a dense cellular network expressing the neural marker β-tubulin III (TUBB33) within the AMOs at d25. f/g Differentiation towards a midbrain fate is further illustrated by widespread expression of Nurr1 and Foxa2 together with TH at day 25. h/i Continuing maturation of AMOs is indicated by the presence of synapses marked by the colocalization of the presynaptic synaptophysin and postsynaptic homer on Map2 positive neurites at day 50 (h, top right corner showing enlargement of two synapses without the Map2 channel) and S100B/GFAP double positive astrocytes at day 75. i Scale bars, 100 μm (a, c, e), 20 μm (b, d, f, g, h, i).

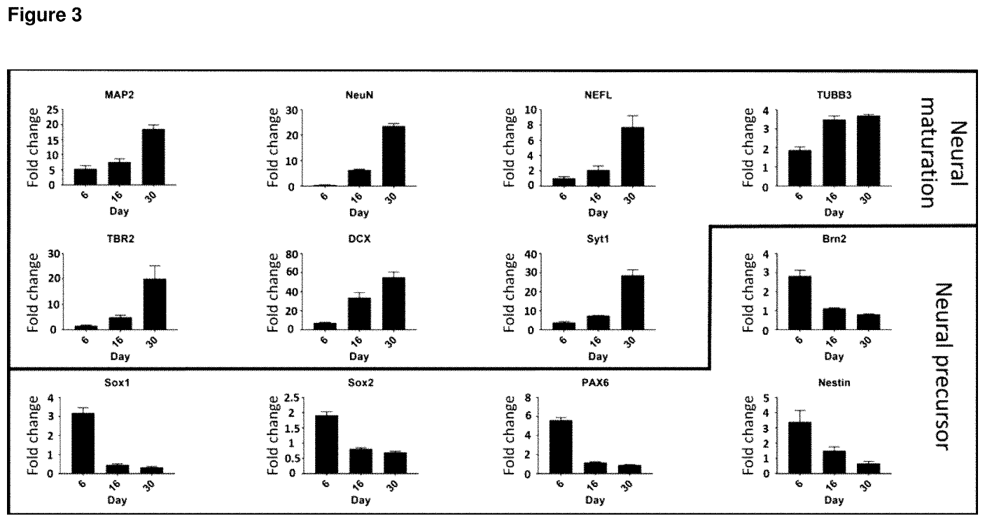

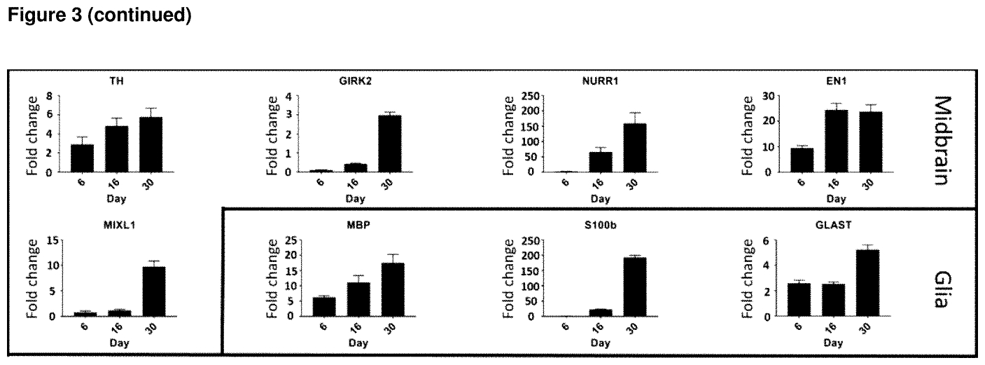

FIG. 3: Quantitative real time PCR shows maturation of AMOs over time
Changes in gene expression during the development of AMOs shown by qPCR. Their continuing maturation is indicated by the increase of neural maturation (MAP2, NeuN, NEFL, TUBB3, TBR2, DCX, Syt1), midbrain (TH, GIRK2, NURR1, EN1, MIXL1), and glia (MBP, S100b, GLAST) as well as the decrease of neural precursor (Brn2, Sox1, Sox2, Pax6, Nestin) markers over time. (n=3 independent differentiations, 2 technical replicates each, error bars=SEM).

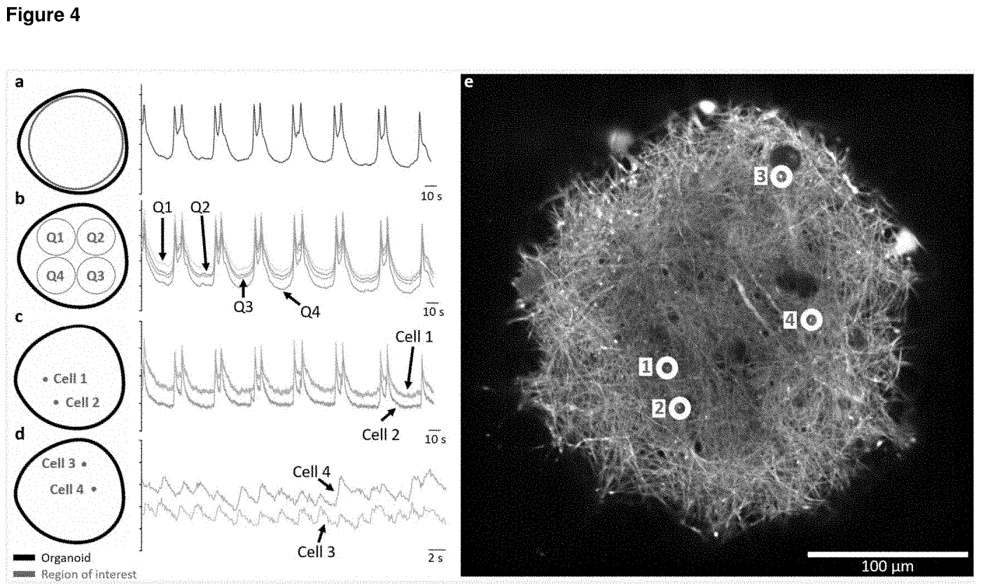

FIG. 4: Calcium imaging reveals spontaneous and synchronized activity throughout entire organoids
a AMOs show spontaneous, organoid-wide spikes of calcium activity. b Division of the optical cross section into quadrants shows that this calcium activity is occurring synchronously throughout the entire organoid. c This synchronous activity pattern can be found down to the level of single cells. d Even distant single cells show additional levels of synchronized activity faster than the organoid-wide spikes. e Single fluorescent confocal slice indicating the position of cells measured in c and d, also illustrating the dense network of active cells. For calcium dynamics, please refer to movie 2. Scale bar=100 μm.

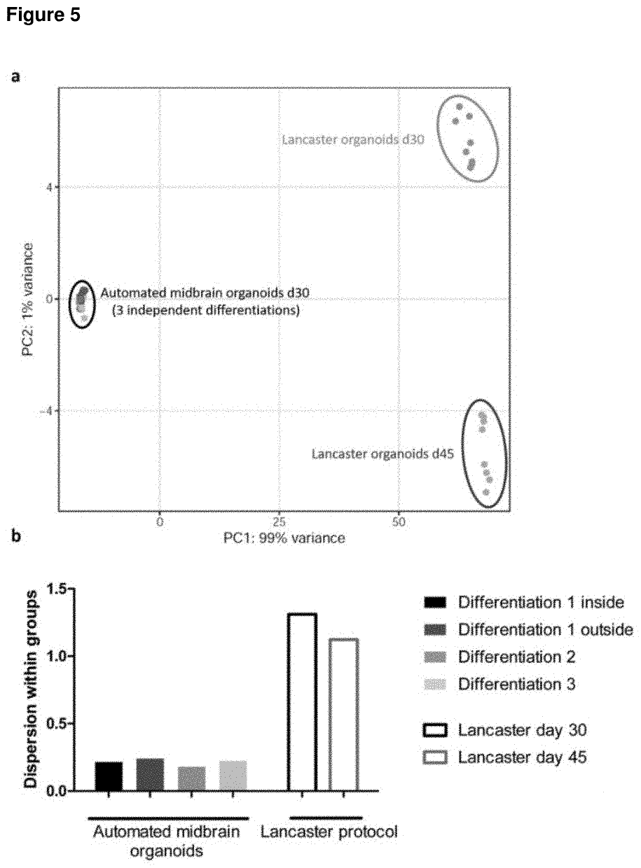

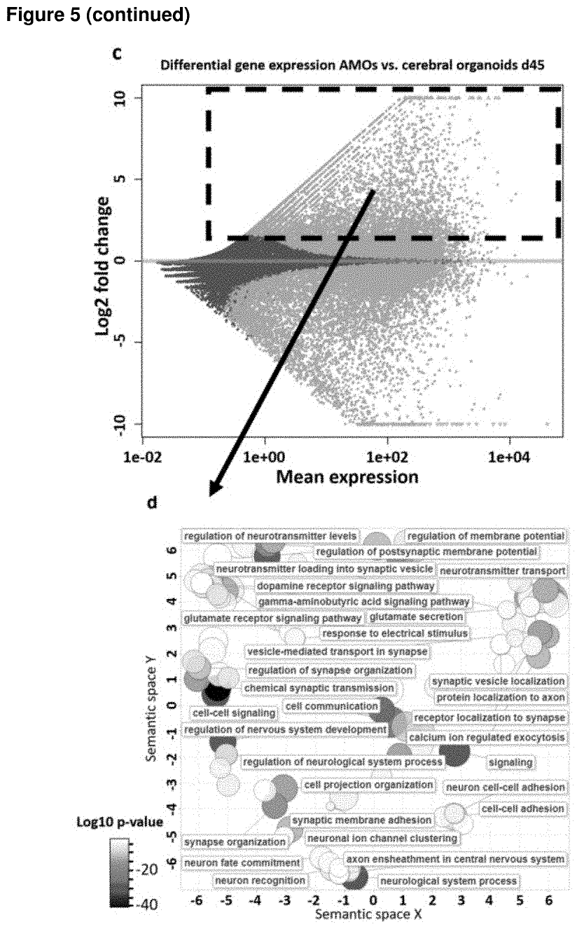

FIG. 5: RNA sequencing reveals less intra- and inter-batch variability in automated midbrain organoids compared to established protocols a AMOs from 3 independent differentiations cluster more closely together than iPSC organoids from one batch derived according to the protocol by Lancaster et al. in a PCA plot based on RNA sequencing data. b Quantification of the dispersion within the different groups of the PCA plot reveals approximately 6 times lower variance for AMOs compared to the iPSC-based Lancaster organoids. There is no apparent difference between organoids from the outside or inside of the plate. n=8 organoids per group, except n_{1 outside}=18 and n_{1 inside}=30. c Plot showing the differential gene expression between day 30 AMOs and day 45 Lancaster organoids. The genes upregulated in AMOs (dotted box) were used for a GO term analysis in d. GO term analysis reveals that most genes upregulated in the AMOs are related to neuronal maturation, especially synaptic activity. Visualization via REVIGO (Supek, F., Bosnjak, M., Skunca, N. & Smuc, T. REVIGO summarizes and visualizes long lists of gene ontology terms. PLoS One 6, e21800 (2011)), grouping GO terms based on semantic similarity. Each GO term is represented by a circle where the circle sizes indicates the number of genes included in the term and colors show the significance of enrichment of the term.

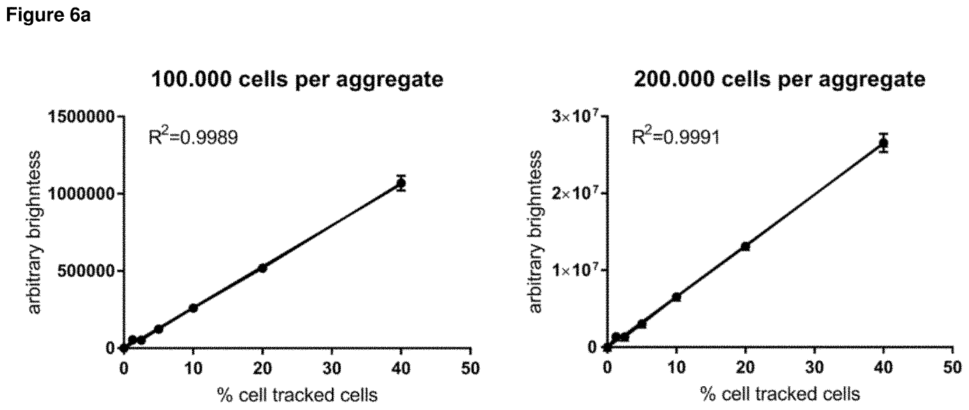

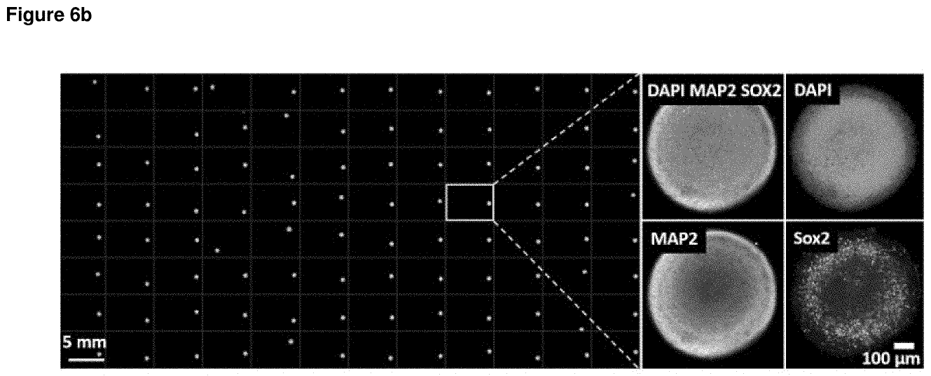

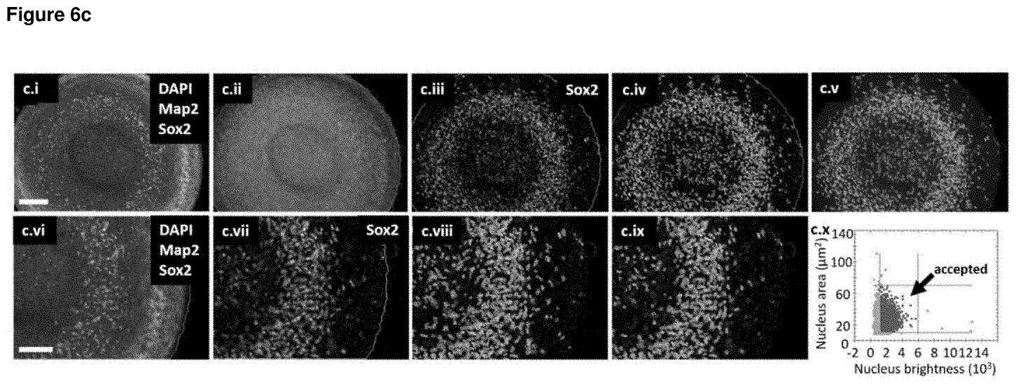

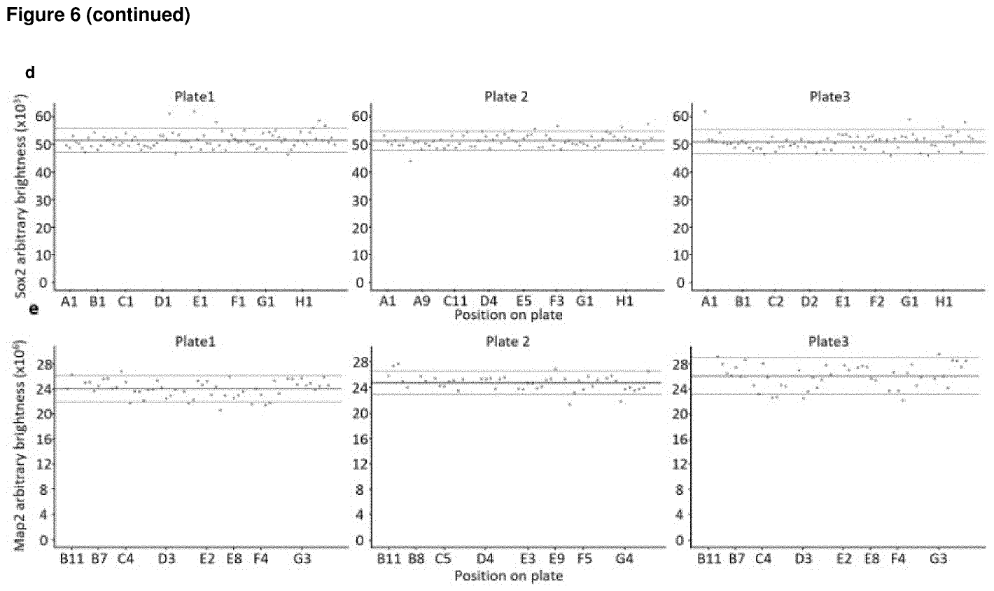

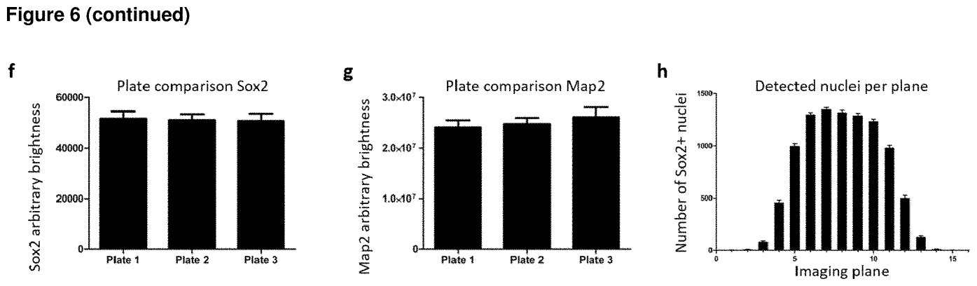

FIG. 6: Automated whole mount immunostaining is quantitative and reveals high homogeneity of automated midbrain organoids
a The optical analysis workflow allows quantification of cell numbers in 3D aggregates. The correlation between the number of fluorescent cells in an aggregate and its brightness measured with our workflow is highly linear (R2>0.99) for large-scale 3D aggregates of different size (100.000 or 200.000 cells per aggregate, diameter>800 um). n=3, error bars=SEM. b Overview of an entire 96 well plate processed with our HTS-compatible optical analysis workflow (left) and an example single plane confocal image of a single organoid illustrating the high cellular resolution achieved with high content imaging (right). Scale bar=100 μm. c Visualization of the automated image analysis sequence for the example of Sox2. Images show a single automatically acquired confocal image plane through the center of an AMO. Top row: Overview, with bottom row providing enlarged view. c i/vi Starting image. c ii All three channels summed for AMO detection. Detected organoid area overlaid in green. c iii/vii Sox2 channel after sliding parabola treatment to remove background. c iv/viii Sox2 channel with detected nuclei. c v Nuclei selected as Sox2+ according to size and brightness (green) and rejected nuclei (red). c ix Selected nuclei from h marked, rejected nuclei unmarked. c x Scatter plot showing nuclear size and brightness distribution and selection thresholds. Scale bars: 100 μm (c i), top row; 70 μm (c vi), bottom row. d-g) AMOs are homogenous with regards to the amount of Sox2 (d/f) and Map2 (e/g) positive cells they contain. In d and e each dot represents a single organoid, each plate originating from an independent differentiation. The continuous line represents the mean brightness (i.e. Map2/Sox2 content) and the dotted lines correspond to 1.5 confidence intervals. f and g summarize the data of the dot plots as a bar graph. (Error bars=standard deviation, SD). h The number of Sox+ nuclei detected in each imaged confocal plane correlates with organoid morphology. The high content image analysis workflow detects many nuclei where the organoid diameter is largest (plane 6-10) and fewer nuclei in the first/last planes where the organoid area is smaller. (Error bars=SEM).

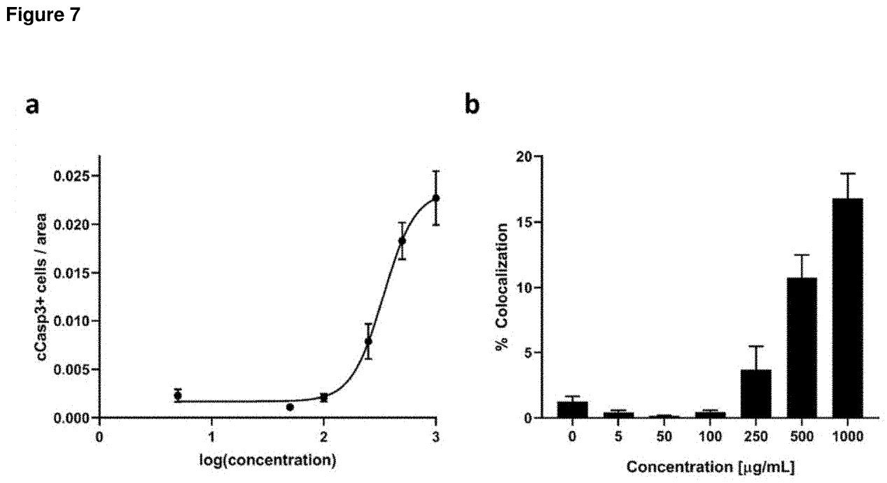

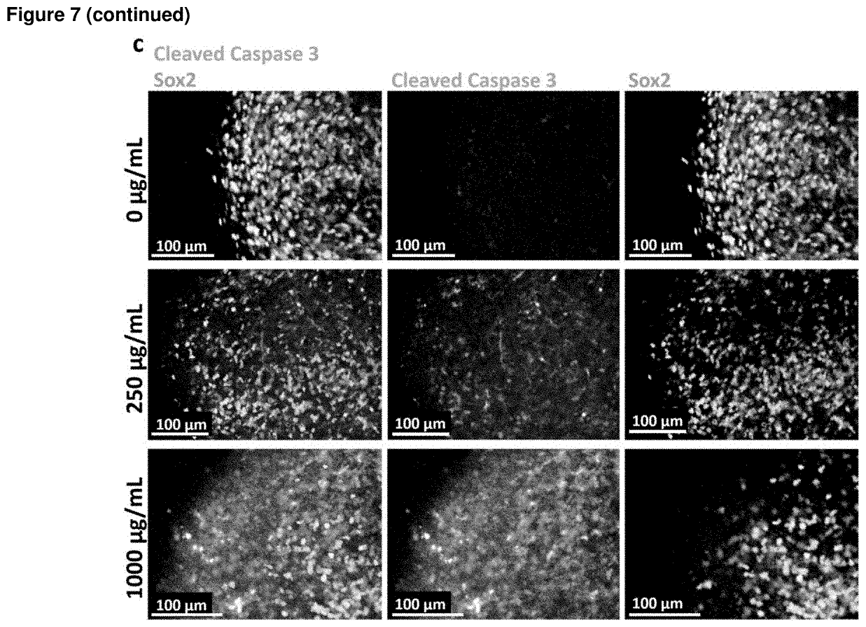

FIG. 7: a) Dose-response curve of typical sigmoidal shape showing an increase in apoptotic cCasp3 positive cells in AMOs with increasing concentrations of G418. Depicted is the total number of cCasp3 cells in an aggregate, normalized by the aggregate area, on the y-axis against the logarithmic concentration of G418 on the x-axis. n3, error bars=SEM b) The cCasp3 signal shows little colocalization with Sox2, indicating that not neuronal precursors but other, more mature cell types are primarily affected by the treatment. The percentage of Sox2+ neural precursors among the apoptotic cells increases with the inhibitor concentration but remains relatively low with a maximum of approximately 15%. n3, error bars=SEM c) Example single plane confocal images generated through the high content optical analysis pipeline illustrating the increase in cCasp3+ apoptotic cells in the AMOs with increasing inhibitor concentrations. We treated AMOs with the indicated concentrations of G418 for 4 days starting at day 50 of culture. Scale bars=100 μm.

FIG. 8: a) Representative pictures of three AMOs from three completely independent batches/differentiations displaying high levels of homogeneity in both morphology and size. b) Whole brain organoids produced by us following the state-of-the-art protocol by Lancaster, M. A. et al. (Cerebral organoids model human brain development and microcephaly. Nature 501, 373-379, doi:10.1038/nature12517 (2013)). Despite optimizing and standardizing the protocol for use with an automatic liquid handling system (pipetting robot) we obtained highly variable 3D structures. c) Whole brain organoids published by Velasco, S. et al. (Individual brain organoids reproducibly form cell diversity of the human cerebral cortex. Nature 570, 523-527, doi:10.1038/s41586-019-1289-x (2019)) also following a modified version of the Lancaster protocol show similar degrees of variability as in b). d) Brain organoids by Mariani et al.³ showing high morphological and size variation.

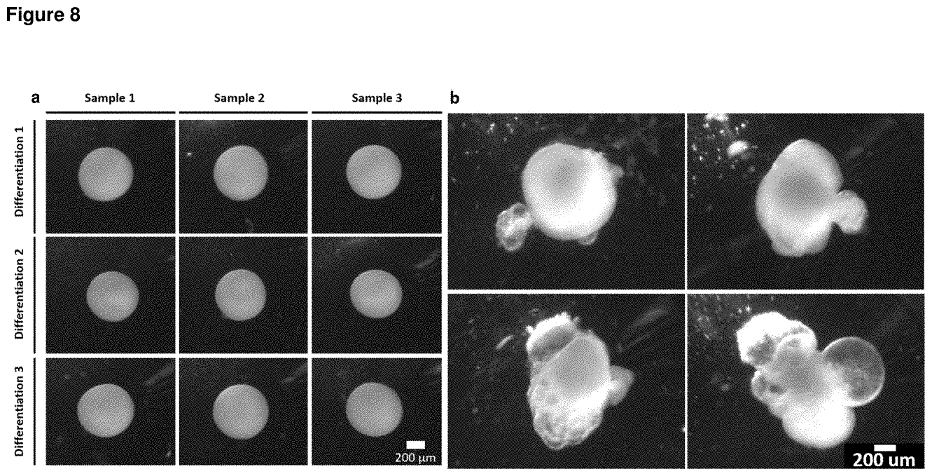

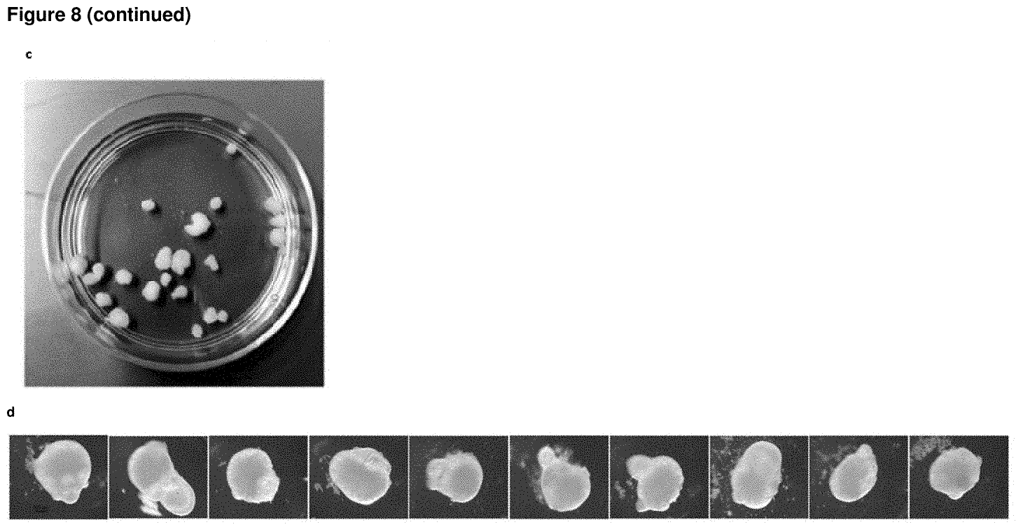

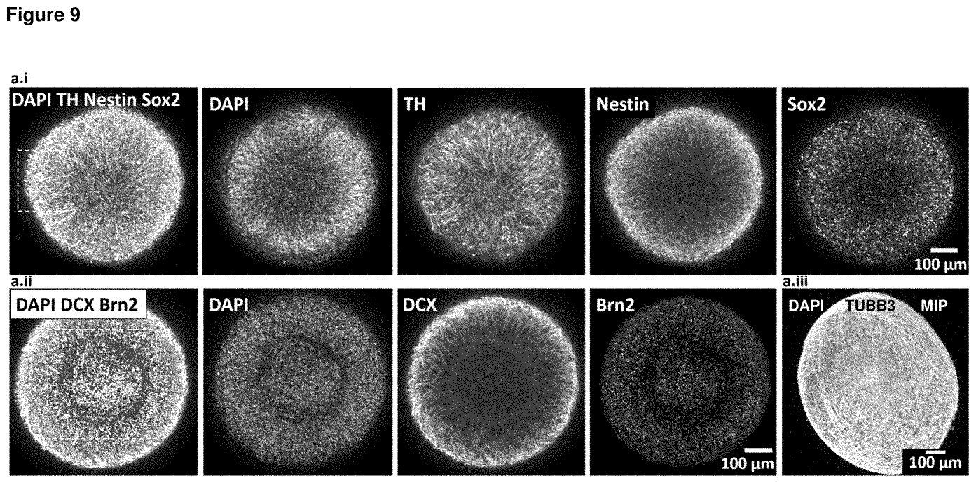

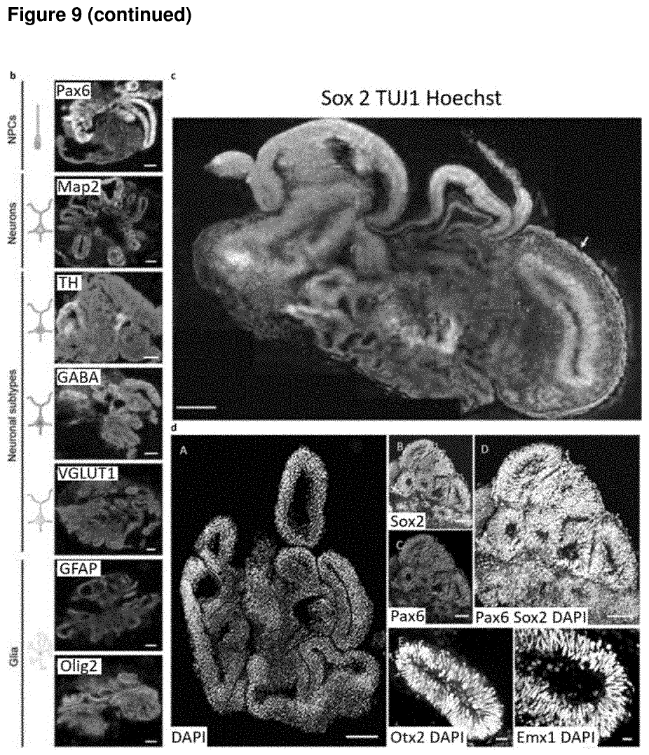

FIG. 9: a) Representative confocal imaging pictures of the internal structure of AMOs displaying a highly ordered, homogeneous and reproducible structure. b) Overview of several whole brain organoids by Quadrato, G. et al. (Cell diversity and network dynamics in photosensitive human brain organoids. Nature 545, 48-53, doi:10.1038/nature22047 (2017)) generated via a modified version of the Lancaster protocol. The organoids are all of the same age yet show a strikingly variable, disorganized and irreproducible structure/internal organization. c) The brain organoid by Lancaster, M. A. et al. (Cerebral organoids model human brain development and microcephaly. Nature 501, 373-379, doi:10.1038/nature12517 (2013)) shows a complex but randomly and irreproducibly organized structure. d) Brain organoids by Krefft, O., et al. (Generation of Standardized and Reproducible Forebrain-type Cerebral Organoids from Human Induced Pluripotent Stem Cells. J Vis Exp, doi:10.3791/56768 (2018)), while being described as “standardized and reproducible”, yet displays heterogeneous and unpredictable structures.

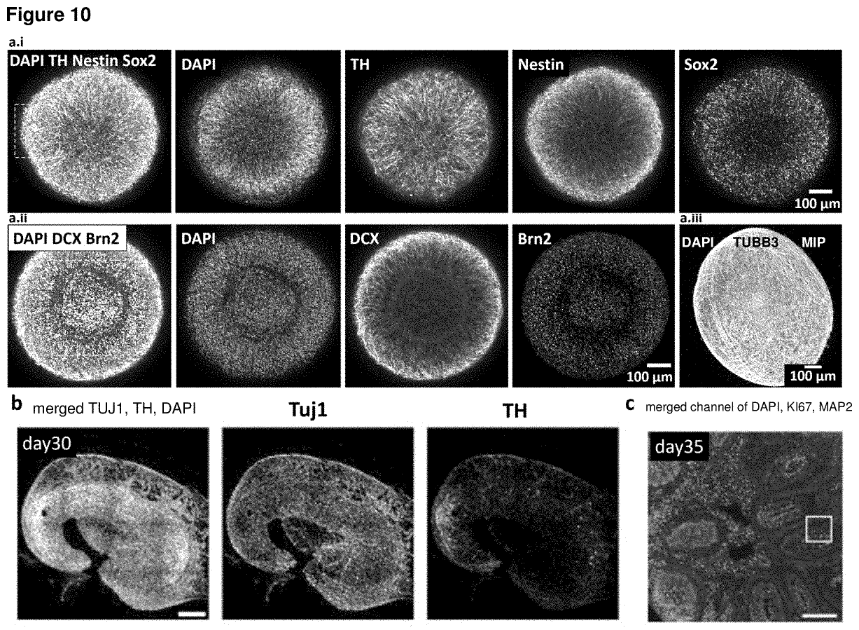

FIG. 10: a) Representative confocal imaging pictures of the internal structure of AMOs displaying a highly ordered, homogeneous and reproducible structure. b) Overview of a midbrain organoid by Monzel, A. S. et al. (Derivation of Human Midbrain-Specific Organoids from Neuroepithelial Stem Cells. Stem Cell Reports 8, 1144-1154, doi:10.1016/j.stemcr.2017.03.010 (2017)), the protocol most similar to ours, displaying a more disorganized structure than AMOs. c) Image of a midbrain organoid by Jo, J. et al. (Midbrain-like Organoids from Human Pluripotent Stem Cells Contain Functional Dopaminergic and Neuromelanin-Producing Neurons. Cell Stem Cell 19, 248-257, doi:10.1016/j.stem.2016.07.005 (2016)), the most well-known published midbrain organoid protocol also showing a non-reproducible structure.

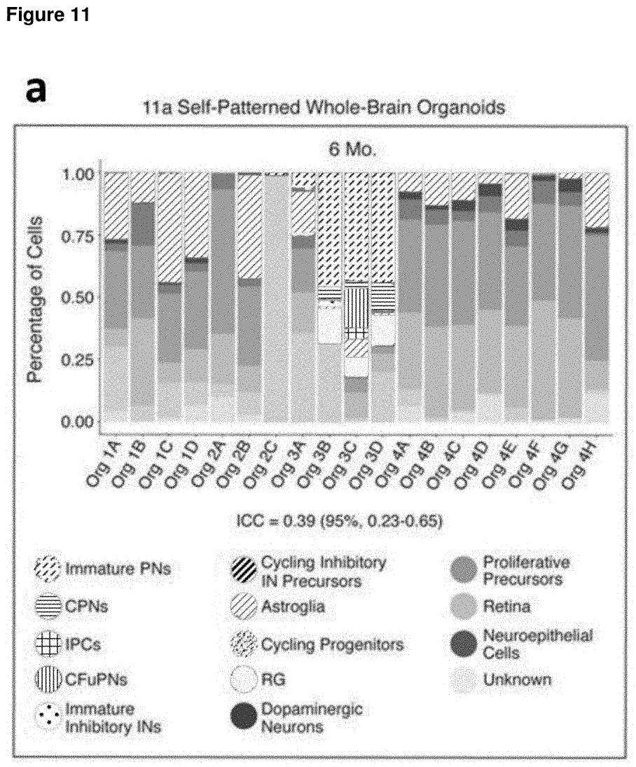

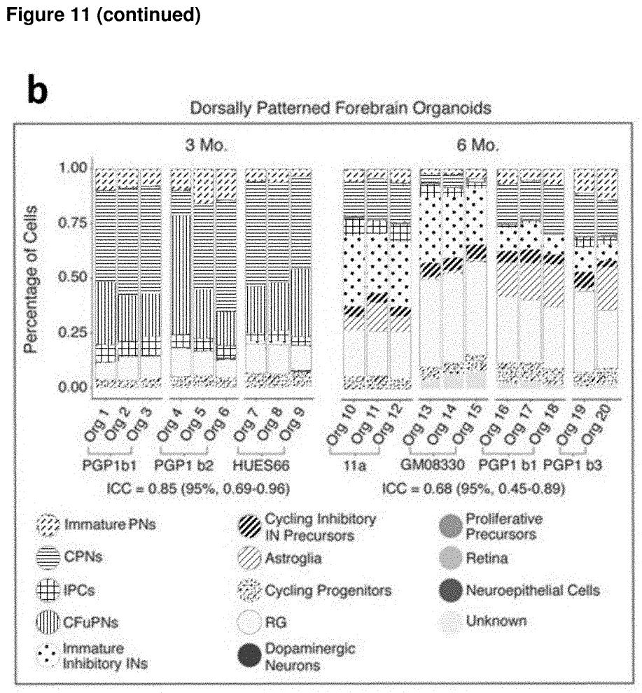

FIG. 11: Here, Velasco, S. et al. (Individual brain organoids reproducibly form cell diversity of the human cerebral cortex. Nature 570, 523-527, oi:10.1038/s41586-019-1289-x (2019)) analyzed the cellular composition of different state-of-the-art/published organoids. a) The self-patterned whole-brain organoids were generated following the Lancaster, M. A. et al. (Cerebral organoids model human brain development and microcephaly. Nature 501, 373-379, doi:10.1038/nature12517 (2013)) method which still represents the most commonly used protocol in the field. The analysis of cellular composition revealed huge heterogeneity between different samples. Especially striking is that some “important” cell types claimed to be present in the organoids (immature PNs, blue bars) are only found in a small number of the tested samples. b) The dorsally patterned forebrain organoids were generated following a modified version of the protocol by Kadoshima, T. et al. (Self-organization of axial polarity, inside-out layer pattern, and species-specific progenitor dynamics in human ES cell-derived neocortex. Proc Natl Acad Sci USA 110, 20284-20289, doi:10.1073/pnas.1315710110 (2013)). Velasco, S. et al. (Individual brain organoids reproducibly form cell diversity of the human cerebral cortex. Nature 570, 523-527, oi:10.1038/s41586-019-1289-x (2019)) claim that those represent a reproducible model. While they are in fact more reproducible than the organoids in a), the graph still shows significant differences between single samples and especially between different cell lines.

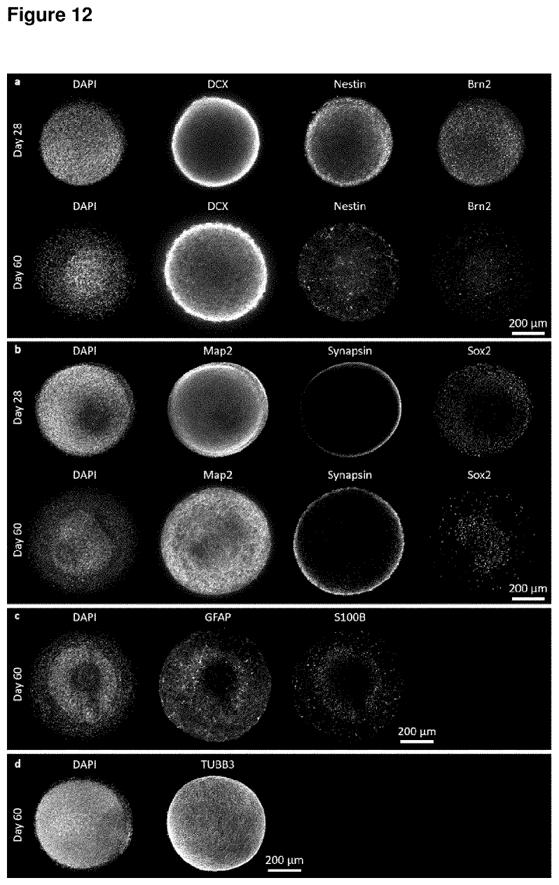

FIG. 12: Single optical confocal slices (a-c) or maximum intensity projections (d) of whole mount stained and cleared non-patterned automated brain organoids (NABOs). a/b) The expression of typical neural (DCX, Map2) and neural precursor (Nestin, Brn2, Sox2) markers is homogeneously distributed throughout the organoids. The presence of synapses is indicated by expression of the synaptic marker Synapsin. Over time, NABOs mature further as indicated by increasing expression of the neural and synaptic markers DCX, Map2, and Synapsin as well as decreasing expression of the precursor markers Nestin, Brn2, and Sox2 from day 28 (upper panel) to day 60 (lower panel). c) At later time points (day 60) NABOs contain a large number of GFAP and S100B positive astrocytes, many of them double positive for both markers. d) Maximum intensity projection of the mature neural marker TUBB3 illustrating the dense network of neurons in the NABOs.

FIG. 13: Non-patterned automated brain organoids (NABOs) are highly homogeneous with regard to their size and morphology. a) Size measurement of 20 individual organoids (area of the largest cross-section). Every dot represents one organoid, the line corresponds to the mean size of all 20 organoids. The size distribution of NABOs shows very little variance with a coefficient of variance of only 3%. b) Representative pictures of 9 NABOs further illustrating their homogeneous size and morphology. Scale bar=200 μm, all organoids shown in a) and b) were cultured for 45 days before analysis.

The Examples illustrate the invention.

### EXAMPLE 1

### Methods

smNPC Culture

All cells and organoids were maintained at 37° C. and 5% CO2 unless otherwise noted. We cultured human small molecule precursor cells (smNPCs) with minor modifications as previously described22. Briefly, we grew smNPCs in 0.0125% (v/v) Matrigel (BD)-coated 6-well plates (Sarstedt) in N2B27 medium supplemented with the small molecules smoothened agonist (0.5 μM, SAG, Cayman Chemical) and CHIR 99021 (3 μM, Axon MedChem). N2B27 consisted of DMEM-F12 (Thermo Fisher) and Neurobasal Medium (Thermo Fisher) at a 1:1 ratio, enriched with 1:400 diluted N2 supplement (Thermo Fisher), and 1:200 diluted B27 supplement without vitamin A (Thermo Fisher), 1% penicillin/streptomycin/glutamine (Thermo Fisher), and 200 μM ascorbic acid (Sigma-Aldrich). Typically, we exchanged medium every other day. The cells were split every 5-7 days at a splitting ratio of 1:10 to 1:20 via accutase treatment (Sigma-Aldrich) for ca. 15 min at 37 C, yielding a single cell solution. To stop the digestion, the cells were diluted in DMEM-F12 with 0.1% BSA (Thermo Fisher) and centrifuged at 1200 g for 2 minutes. The cell pellet was resuspended in fresh smNPC medium (N2B27 with SAG and CHIR) and plated on Matrigel-coated 6-well plates.

### AMO Generation

After digestion by accutase, we seeded 9000 smNPCs in each well of a conical 96-well plate (Thermo Fisher) in smNPC medium and allowed them to aggregate for 2 days. To increase inter-cell adhesion, we added 0.4% (w/v) polyvinyl alcohol (PVA). Starting at day 2, cells undergo ventral patterning over 4 days in 2 feedings by removal of CHIR99021 in the continued presence of SAG. The addition of 0.5 ng/mL brain derived neurotrophic factor (BDNF, PeproTech) and 1 ng/mL glial cell line-derived neurotrophic factor (GDNF, PeproTech) boost maturation and cell survival during the rest of the neural maturation. After ventralization, we removed SAG on day 6, further supported midbrain differentiation and maturation by the addition of 0.5 ng/mL transforming growth factor beta 3 (TGF8-3), and 100 μM dibutyryl cyclic adenosine monophosphate (dbcAMP, Sigma-Aldrich). A single dose of 5 ng/mL Activin A was added on day 6 only. Depending on the desired degree of maturity, the duration of the maturation phase can be prolonged to 100 days and longer.

### Generation of NABOs

Like the automated midbrain organoids, the non-patterned automated brain organoids were generated, maintained and analyzed in a fully automated fashion. The principle workflow remains identical, only media formulations and media timings differ, demonstrating the flexibility of the method of the invention to accommodate generation of a variety of different, preferably neural structures. In short, the method for generating the NABOs is as follows:

After digestion by accutase, we seeded 9000 smNPCs in each well of a conical 96-well plate (Thermo Fisher) in smNPC medium and allowed them to aggregate for 2 days. To increase inter-cell adhesion, we added 0.4% (w/v) polyvinyl alcohol (PVA) to the seeding medium. The smNPC medium is based on N2B27 medium supplemented with the small molecules smoothened agonist (0.5 μM, SAG, Cayman Chemical) and CHIR 99021 (3 μM, Axon MedChem). N2B27 medium consisted of DMEM-F12 (Thermo Fisher) and Neurobasal Medium (Thermo Fisher) at a 1:1 ratio, enriched with 1:400 diluted N2 supplement (Thermo Fisher), and 1:200 diluted B27 supplement without vitamin A (Thermo Fisher), 1% penicillin/streptomycin/glutamine (Thermo Fisher), and 200 μM ascorbic acid (Sigma-Aldrich). Starting at day 2, the aggregates undergo undirected neural differentiation by withdrawal of SAG and CHIR from the medium and addition of 1 ng/mL brain derived neurotrophic factor (BDNF, PeproTech) and 1 ng/mL glial cell line-derived neurotrophic factor (GDNF, PeproTech). These growth factors boost maturation and cell survival during the rest of the neural maturation. Depending on the desired degree of maturity, the duration of the maturation phase can be prolonged to 100 days and longer. For analysis, organoids were whole mount stained and optically cleared as disclosed herein.

### Size Measurement of Organoids

For size measurements of both AMOs and NABOs, we took brightfield images of randomly selected organoids using a stereo microscope (Leica MZ10 F, camera: Leica DFC425 C). Images were processed with ImageJ/Fiji (Schindelin, J. et al. Fiji: an open-source platform for biological-image analysis. Nat Methods 9, 676-682 (2012)) using a custom-tailored standardized workflow. The auto threshold function was used to discriminate organoids from the background followed by a measurement of their area with the analyze particles function. The measured area corresponds to the largest cross-section of the organoid. Data were outputted to Microsoft Excel and GraphPad Prism v7.0 (Graphpad Software, Inc.) for further analysis.

### Whole Mount Staining and Clearing

In order to analyze protein expression in 3D in a HTS-compatible manner, we adapted a whole mount staining protocol based on Lee et al. ACT-PRESTO: Rapid and consistent tissue 579 clearing and labeling method for 3-dimensional (3D) imaging. Sci Rep 6, 18631 (2016) for organoids and optimized it for use in an automated liquid handling system. After fixation with 4% PFA (VWR) for 10-15 minutes, we stained the organoids with primary and secondary antibodies (Alexa Fluor secondary antibodies, Thermo Fisher) for 6 days each. We diluted the antibodies in a blocking and permeabilization solution (6% BSA, 0.5% Triton-X 100 (Roth), 0.1% (w/v) sodium azide (Sigma-Aldrich) in PBS (Sigma-Aldrich)) and renewed it every 2 days. Between primary and secondary antibody incubation as well as after the staining procedure we washed the organoids 5 times for 1 h with 0.1% Triton X-100 in PBS. This extremely long staining procedure allows the antibodies to fully penetrate the organoids despite their large size and high density. To enable full penetration by microscope illumination, the whole mount staining procedure is followed by BABB-based tissue clearing Dent, J. A., Polson, A. G. & Klymkowsky, M. W. A whole-mount immunocytochemical analysis of the expression of the intermediate filament protein vimentin in Xenopus. Development 105, 61-74 (1989). First, the organoids were dehydrated stepwise through a methanol (Roth) series (25%, 50%, 70%, 90%, 100%, 15 minutes each). Next, they were transferred to an organic solvent-resistant cyclo-olefin 96-well plate (“Screenstar”, Greiner Bio-One). The samples were incubated for 30 minutes in 1:1 methanol/389 BABB (benzyl benzoate (Sigma-Aldrich) and benzyl alcohol (Sigma-Aldrich) 1:1) and subsequently kept in BABB for imaging. We used Imaris v8.4 (Bitplane, Oxford Instruments) for 3D rendering of confocal slices (FIG. 1d).

### Quantitative Real Time PCR

We performed RNA isolation for quantitative real time PCR (qPCR) analysis using the NucleoSpin RNA XS kit (Macherey-Nagel) according to the manufacturer's instructions. Depending on their age, we pooled 32 (d6), 24 (d16), or 18 (d30) organoids from one batch in order to yield enough RNA for downstream analysis. We determined RNA concentration and purity using a NanoDrop 8000 spectrophotometer (Thermo Fisher) and performed reverse transcription according to standard protocols using 1000 ng RNA per reaction. For quantification of gene expression, we used the Biomark 48.48 integrated fluidic circuit (IFC) Delta Gene assay (Fluidigm) according to the manufacturer's instructions. Briefly, following 14 cycles of preamplification, the samples were subjected to an exonuclease I (New England Biolabs) treatment (37° C. for 30 min and 80° C. for 15 min) and diluted twentyfold with DNA Suspension buffer (TEKnova). The samples (in duplicates) and assay mixtures were loaded onto a 48.48 microfluidic ICF chip and run on the BioMark real-time PCR reader (Fluidigm) where they were amplified and measured according to manufacturer's instructions. Data analysis was performed using the BioMark real-time PCR analysis software 4.3.1 (Fluidigm) standard settings. Data was transferred to Microsoft Excel for further processing and GraphPad Prism v7.0 for plotting. GAPDH served as housekeeping gene.

### Calcium Imaging

For calcium imaging, we added 10 μM cell-permeant Fluo-4 AM (Thermo Fisher) diluted in organoid medium to the organoids and incubated for 60 minutes at 37° C. Imaging was performed using a Dragonfly spinning disc confocal microscope (Andor, Oxford Instruments) at a frequency of 10 Hz for 4 minutes. Data analysis was performed using ImageJ/Fiji (Schindelin, J. et al. Fiji: an open-source platform for biological-image analysis. Nat Methods 9, 676-682 (2012)). First, different ROls were defined as depicted in FIG. 4. Then, the mean fluorescence intensity in those ROls was measured over time and plotted using GraphPad Prism v7.0. The video was assembled via ImageJ/Fiji (Schindelin, J. et al. Fiji: an open-source platform for biological-image analysis. Nat Methods 9, 676-682 (2012)) and the frame rate accelerated to compress 4 minutes real time at 10 Hz into 20 seconds running time.

iPSC Culture

Human iPSC culture was performed feeder-free using modified FTDA medium (Frank, S., Zhang, M., Scholer, H. R. & Greber, B. Small molecule-assisted, line-independent maintenance of human pluripotent stem cells in defined conditions. PLoS One 7, e41958 (2012)) in 0.0125% (v/v) Matrigel-coated 6-well plates with a previously described healthy control line. FTDA medium consisted of DMEM-F12 supplemented with 1% human serum albumin (Biological Industries), 1% Chemically Defined Lipid Concentrate (Life Technologies), 0.1% Insulin-Transferrin-Selenium (BD), 1% penicillin/streptomycin/glutamine. We fed the iPSCs daily and added 10 ng/mL FGF2 (PeproTech GmbH), 0.2 ng/mL TGFβ3 (PeproTechGmbH), 50 nM Dorsomorphin (Santa Cruz), 5 ng/mL Activin A (eBioscience), 20 nM C59 (Tocris) before each media exchange. We split the iPSCs as single cells every 3-5 days using accutase for ca. 10 minutes at 37° C. We transferred 600.000 cells per well of a 6-well plate to be seeded to DMEM-F12 with 0.1% BSA and centrifuged at 1200 g for 2 minutes. We resuspended the cell pellet in fresh FTDA medium supplemented with 1:2000 ROCK inhibitor Y-27632 (tebu-bio) and plated the iPSCs on Matrigel-coated 6-well plates.

iPSC-Based Organoid Culture

For iPSC-derived organoid generation we followed the protocol by Lancaster et al. (Cerebral organoids model human brain development and microcephaly. Nature 501, 373-379 (2013)) with minor modifications. Briefly, we dissociated iPSCs to single cells by accutase treatment and plated 9000 cells per well in a conical 96 well plate in low FGF stem cell medium (DMEM-F12 with knockout serum replacement (KOSR, Thermo Fisher) 1:5, fetal bovine serum (Biochrom) 1:33.3, 1% penicillin/streptomycin/glutamine, 1% non-essential amino acids (NEAA, Thermo Fisher), β-mercaptoethanol (Thermo Fisher) 1:143, 4 ng/μL FGF2, 50 μm ROCK inhibitor Y-27632, and 0.4% PVA on seeding day only to facilitate aggregation). We exchanged the medium every other day, FGF2 and Y-27632 were withdrawn on day 6. Neural induction was started on day 8 (neural induction medium: DMEM-F12 with KOSR 1:5, 1% penicillin/streptomycin/glutamine, 1% 439 non-essential amino acids, N2 supplement 1:100, and Heparin (Sigma-Aldrich) 1 μg/mL) and continued for 6 days with media changes every other day. On day 13, we embedded the aggregates into 30 μL matrigel droplets and transferred them to 6 cm2 suspension tissue culture dishes (Sarstedt) in cerebral organoid differentiation medium (DMEM-F12 and Neurobasal 1:1 with 1% penicillin/streptomycin/glutamine, 1% NEAA, N2 supplement 1:200, B27 supplement without vitamin A 1:100, Insulin (Sigma-Aldrich) 1:4000, and β-mercaptoethanol 1:285714). We placed the culture dishes on a shaker at 37° C. and 5% CO2 and fed the organoids every other day. On day 20 the B27 supplement was replaced by B27 with Vitamin A (Thermo Fisher) and organoids were cultured until day 30/45.

### RNA Sequencing

To isolate RNA of single organoids we used the Direct-zol-96 RNA kit (Zymo Research) according to the manufacturer's instructions. We assessed RNA concentration and purity using a NanoDrop 8000 spectrophotometer and RNA integrity with a Bioanalyzer (Agilent Technologies) per standard protocols. Next, mRNA was enriched using the NEBNext Poly(A) Magnetic Isolation Module (NEB) followed by strand-specific cDNA NGS library preparation (NEBNext Ultra II Directional RNA Library Prep Kit for Illumina, NEB). The size of the resulting library was controlled by use of a D1000 ScreenTape (Agilent 2200 TapeStation) and quantified using the NEBNext Library Quant Kit for Illumina (NEB). Equimolar pooled libraries were sequenced in a single read mode (75 cycles) on the NextSeq 500 System (Illumina) using v2 chemistry yielding in an average QScore distribution of 95%>=Q30 score and subsequent demultiplexed and converted to FASTQ files by means of bcl2fastq v2.20 Conversion software (Illumina).

### RNA Sequencing Analysis

We aligned the RNA sequencing reads to the human genome hg19 with TopHat2 aligner (v2.1.1)46, using default input parameters. Gene annotation from Ensembl (version GRCh37.87) were used in the mapping process. The number of reads that were mapped to each gene was counted using the Python package HTSeq (v0.7.2) (Anders, S., Pyl, P. T. & Huber, W. HTSeq—a Python framework to work with high-throughput sequencing data. Bioinformatics 31, 166-169 (2015)) with “htseq-count—mo 464 de union—stranded no”. Principal component analysis and differential expression analysis was performed with raw counts using the R package DESeq2 (v1.18.1). Dispersion within groups was calculated using the average distance between data points and centroids. Genes were considered as deregulated if |log 2FC|>2 and FDR<0.05 using Benjamini-Hochberge multiple test adjustment (Benjamini, Y. & Hochberg, Y. Controlling the False Discovery Rate: A Practical and Powerful Approach to Multiple Testing. Journal of the Royal Statistical Society. Series B (Methodological) 57, 289-300 (1995)). Gene Ontology (GO) term enrichment was analyzed with the bioinformatics web server Gorilla (Eden, E., Navon, R., Steinfeld, I., Lipson, D. & Yakhini, Z. GOrilla: a tool for discovery and visualization of enriched GO terms in ranked gene lists. BMC Bioinformatics 10, 48 (2009) and visualized with REViGO40. All RNA sequencing data was deposited to NCBI GEO database.

### Quantification of Whole Mount Staining and Clearing

To assess how quantitative our imaging workflow is, we performed a dilution experiment. We mixed unlabeled smNPCs with different percentages (1.25%, 2.5%, 5%, 10%, 20%, 40%) of CellTracker deep red dye (Life technologies)-labeled cells (labeling according to standard protocols, dye concentration 1:20000) and aggregated them in smNPC maintenance medium with 0.4% PVA. To explore the effects of overall aggregate size on quantitation, we generated aggregates with 100.000 as well as 200.000 cells in total. After 1 day of aggregation, the aggregates were fixed with 4% PFA, subjected to BABB-based tissue clearing, imaged, and analyzed as described below.

### High Content Imaging and Analysis

After staining and clearing, we achieved uniform aggregate positioning within the wells by tilting the plates off the horizontal plane at 60 degrees for 1 minute. Image acquisition was carried out in an Operetta high content imager (Perkin Elmer) and images were analyzed in Harmony 4.1 software. We acquired a total of 16 confocal planes in three channels (DAPI, Sox2-488, and MAP2-647) with an inter-plane spacing of 36.6 μm for a total stack of 549 μm, covering the entire organoid height. To define the organoid region on each image plane, all three channels were summed, filtered with a median filter to remove small localized features and bright areas were identified via the “find image region” function. After cleaning the edge of the organoid region by dilation and erosion steps of 10 and 3 pixels, respectively, we identified bona fide organoids by selecting for regions with a minimum of 300 arbitrary brightness units (abu) and 4000 μm2 size. In order to better isolate Sox2+ nuclei from the general background, we ran a sliding parabola algorithm with a curvature setting of 2 across each image plane in the 488 channel. Nuclei were then identified within each organoid region via the “find nuclei” function, algorithm “M” and further selected to be Sox2+ if they were larger than 10 um2 and brighter than 1200 abu. We excluded image artifacts, small dust particles, and overlapping nuclei by omitting nuclei brighter than 6000 abu and larger than 70 μm2 from further quantification. For final output, the number and total brightness of nuclei in 488 and of organoid regions in 647 were summed for all planes and all fields of view in each well and transferred to Microsoft Excel and TIBCO Spotf ire for further annotation, analysis and plotting. We omitted data from wells that contained dust particles, incompletely imaged organoids due to improper positioning, or organoids that have been damaged or lost during culture or downstream processing. Plate 1, 2, and 3 represent independent differentiations of separately thawed and cultured cells of the same freezing batch.

### Electrophysiological Analysis of Single Cells by Patch-Clamping

Due to the morphology of AMOs (high optical density and the fact that most cell bodies are located in a depth of at least 10-20 μm), it was technically impossible to perform the patch-clamp measurements on intact aggregates. Therefore, the organoids were treated with 1 mg/ml trypsin and then mechanically dispersed to obtain single cells. These were seeded on PDL-coated coverslips and cultured for 1-3 days in AMO medium (we stated the age of AMOs at the time of dissociation). The transmembrane currents were recorded from isolated cells using the whole-cell configuration of the patch-clamp technique (Hamill, O. P., Marty, A., Neher, E., Sakmann, B. & Sigworth, F. J. Improved patch-clamp techniques for high-resolution current recording from cells and cell-free membrane patches. Pflugers Arch 391, 85-100 (1981)). The patch pipettes were fabricated from borosilicate glass on a Sutter P1000 (Sutter Instrument company) pipette puller. When filled with pipette solution, they had a tip resistance of 4-6 MΩ. Recordings were done using an EPC-10 amplifier (HEKA Elektronik) and Patchmaster aqusition software (HEKA Elektronik). Series resistance, liquid junction potential, pipette and whole-cell capacitance were cancelled electronically. Bath solution contained (mM): NaCl 140, KCl 2.4, MgCl2 1.2, CaCl2 2.5, HEPES 10, D-glucose 10, pH 7.4 and the pipette solution contained (mM): K-aspartate 125, NaCl 10, EGTA 1, MgATP 4, HEPES 10, D-glucose 10, pH 7.4 (KOH). We performed all experiments at room temperature. Recordings of current-voltage relationship (I-V curves) were done in voltage-clamp mode at a holding potential of −70 mV. Recordings of evoked action potentials were performed in current-clamp mode. Data were analyzed using Patcher's Power Tool routine for IgorPro (WaveMetrics), SciDAVis (http://scidavis.sourceforge.net/) and Origin Pro 2019 (Origin Lab). To reveal the shape of I-V curves, single traces were normalized to the peak amplitude and then averaged.

### 3D Toxicology Assay

At day 50 we treated AMOs with increasing concentrations (0, 5, 50, 100, 250, 500, 1000 μg/mL) of G418 added directly to the culture medium. After 2 days, we renewed the medium (including identical inhibitor concentrations) and fixed the aggregates after a total of 4 days of treatment. Fixation, whole mount immunostaining for cCasp3 and Sox2 as well as BABB-based clearing was performed as above. Image analysis followed the steps as outlined in the high content analysis section with slight modifications to accommodate the individual brightness, morphology, and background characteristics of the cCasp3 staining. Briefly, after identifying AMOs and Sox2+ cells as described previously, the cCasp3 channel was background corrected by running a sliding parabola algorithm with a curvature setting of 10 across each confocal slice of the AMO. We identified apoptotic cells via the “find nuclei” function in the 647 channel, algorithm “M” and further selected them to be cCasp3+ if they were larger than 11 μm2, smaller than 100 μm², and brighter than 2700 abu. We considered cells to be Sox2/cCasp3 double-positive if they fulfilled the criteria for both filters at the same time. The results were outputted to Microsoft Excel, reformatted and then transferred to GraphPad Prism v8.0.2 for plotting, data analysis, and 1050 calculation.

### EXAMPLE 2

### Results

### Automation Enables High Throughput Compatible Production of Homogenous Midbrain Organoids

Screening applications require biological systems that operate within predictable physiological parameters. In order to limit cellular heterogeneity during differentiation, we produced human neural midbrain organoids starting from pluripotent stem cell (PSC)-derived small molecule neural precursor cells (smNPCs) (Reinhardt, P. et al. Derivation and expansion using only small molecules of human neural progenitors for neurodegenerative disease modeling. PLoS One 8, e59252 (2013)). The neural-restricted developmental potential of these cells still allows for the self-organization required for organoid formation (Di Lullo, E. & Kriegstein, A. R. The use of brain organoids to investigate neural development and disease. Nat Rev Neurosci 18, 573-584 (2017); and Monzel, A. S. et al. Derivation of Human Midbrain-Specific Organoids from Neuroepithelial Stem Cells. Stem Cell Reports 8, 1144-1154 (2017)) but leads to more homogenous organoids compared to PSCs-based protocols. Surprisingly, matrigel embedding turned out to be dispensable and reduces batch-to-batch variability matrigel embedding as do standardized mechanical stresses by using an automated liquid handling system (ALHS). While the resulting automated midbrain organoids (AMOs) are structurally less complex than PSC-derived aggregates, they show little intra- and inter-batch variability in size distribution (FIG. 1b, average coefficient of variation (CV) within one batch 3.56%; min 2.2%, max 5.6%), morphology (FIG. 1c), and cellular composition and organization (FIG. 2), making them ideal for HTS-approaches. Furthermore, our workflow generates one organoid per well, maintained independently from other organoids, thus minimizing batch effects due to paracrine signaling observed in bioreactor-based strategies (Quadrato, G. et al. Cell diversity and network dynamics in photosensitive human brain organoids. Nature 545, 48-53 (2017)). If paracrine signaling is desired, our workflow can also accommodate several organoids per well.

### Automated Midbrain Organoids Express Typical Neural and Midbrain Markers and Show Structural Organization

In order to characterize protein localization in our large scale AMOs (>500 μm diameter) and asses the efficiency of their neural/midbrain differentiation at a cellular resolution and in a HTS-compatible manner, we adapted an extended 3D staining protocol by Lee et al. ACT-PRESTO: Rapid and consistent tissue 579 clearing and labeling method for 3-dimensional (3D) imaging. Sci Rep 6, 18631 (2016) for the use in organoids and combined it with benzyl alcohol and benzyl benzoate (BABB)-based tissue clearing (Dent, J. A., Polson, A. G. & Klymkowsky, M. W. A whole-mount immunocytochemical analysis of the expression of the intermediate filament protein vimentin in Xenopus. Development 105, 61-74 (1989)). We found that BABB-based clearing proved to be both the fastest and most efficient method in a comparison of different clearing protocols. The combination of whole mount staining and clearing allows the 3D reconstruction of entire organoids via confocal imaging and enables further detailed 3D quantification and analysis, for example tracing of neurites throughout the whole organoid, which cannot be performed using typical tissue sectioning procedures (see FIG. 1d).

The immunostaining results are depicted as either single confocal optical slices (FIG. 2a, b, c, d, f, g, h, j) or maximum intensity projections (MIP, FIG. 2e). Already at day 25, the AMOs contain large numbers of neurons as indicated by the expression of Map2 (Shafit-Zagardo, B. & Kalcheva, N. Making sense of the multiple MAP-2 transcripts and their role in the neuron. Mol Neurobiol 16, 149-162 (1998)). (FIG. 1d), β-tubulin III (TUBB3) (Leandro-Garcia, L. J. et al. Tumoral and tissue-specific expression of the major human beta tubulin isotypes. Cytoskeleton (Hoboken) 67, 214-223 (2010)) (FIG. 2e), and doublecortin (Gleeson, J. G., Lin, P. T., Flanagan, L. A. & Walsh, C. A. Doublecortin is a microtubule-associated protein and is expressed widely by migrating neurons. Neuron 23, 257-271 (1999)) (DCX, FIGS. 2c and 2d). Presence of tyrosine hydroxylase (TH, FIGS. 2a and 2b), the rate limiting enzyme in dopamine synthesis (Nagatsu, T. Tyrosine hydroxylase: human isoforms, structure and regulation in physiology and pathology. Essays Biochem 30, 15-35 (1995)), as well as the expression of the transcription factors Nurr1 and Foxa2 (Hegarty, S. V., Sullivan, A. M. & O'Keeffe, G. W. Midbrain dopaminergic neurons: a review of the molecular circuitry that regulates their development. Dev Biol 379, 123-138 (2013)) (FIGS. 2f and 2g) are consistent with dopaminergic midbrain differentiation of our organoids. As commonly seen in all neural organoids, AMOs retain a population of neural precursors identified by the ex 92 pression of Sox2 (Ellis, P. et al. SOX2, a persistent marker for multipotential neural stem cells derived from embryonic stem cells, the embryo or the adult. Dev Neurosci 26, 148-165 (2004)) (FIG. 1d, FIGS. 2a and b), Brn2 (Dominguez, M. H., Ayoub, A. E. & Rakic, P. POU-III transcription factors (Brn1, Brn2, and Oct6) influence neurogenesis, molecular identity, and migratory destination of upper-layer cells of the cerebral cortex. Cereb Cortex 23, 2632-2643 (2013)) (FIGS. 2c and d) and the more general neural marker nestin (Hendrickson, M. L., Rao, A. J., Demerdash, O. N. & Kalil, R. E. Expression of nestin by neural cells in the adult rat and human brain. PLoS One 6, e18535 (2011)) (FIGS. 2a and 2b).

Over time, AMOs mature further. At day 50, expression of the presynaptic marker synaptophysin and postsynaptic marker homer (Tadokoro, S., Tachibana, T., Imanaka, T., Nishida, W. & Sobue, K. Involvement of unique leucine-zipper motif of PSD-Zip45 (Homer 1c/ves1-1 L) in group 1 metabotropic glutamate receptor clustering. Proc Natl Acad Sci USA 96, 13801-13806 (1999)), frequently colocalizing with each other on Map2 positive neuritis (FIG. 2h), indicates the presence of synapses. Since gliogenesis follows neurogenesis in vivo (Miller, F. D. & Gauthier, A. S. Timing is everything: making neurons versus glia in the developing cortex. Neuron 54, 357-369 (2007)), we expect the emergence of astrocytes after the initial formation of neurons. Consistently, AMOs contain GFAP and S100b double-positive astrocytes (Gotz, M., Sirko, S., Beckers, J. & Irmler, M. Reactive astrocytes as neural stem or progenitor cells: In vivo lineage, In vitro potential, and Genome-wide expression analysis. Glia 63, 1452-1468 (2015)) at day 75 (FIG. 2j).

In general, the different cell types within the AMOs (i.e. neurons, astrocytes, and neural progenitors) do not form localized structures such as neural rosettes, but are rather organized in four concentric zones around the center of the AMOs (FIGS. 2a and 2c). The outermost zone 4 contains few nuclei with a dense, circumferentially oriented layer of TH+/nestin+/DCX+ cell processes. Cellular orientation changes in the underlying zone 3 closer to the organoid core, with TH+ dopaminergic and DCX+ neurons showing a clear radial organization (FIGS. 2b and 2c). Zone 2, separating this region of radially organized neurons and the core, contains circumferentially oriented DCX+ neurons and few Brn2+ neural precursors (FIGS. 2c and 2d). The core itself, zone 1, includes mostly neural precursors and few neurons. Within a given distance from the center, the different cell types are homogeneously distributed around the entire radius of the microtissues. In the context of HTS compatibility, this radial symmetry is an advantage over protocols yielding more complex, yet more heterogeneous organoids with locally randomly divergent sub-domains as it renders optical quantification independent of the orientation of the microtissues in the well.

Ultrastructural analysis of AMOs (Figure S2) supports the immunofluorescence data revealing a dense 3D cell architecture consistent with neuronal cell bodies surrounded by nerve fibers. Analyzing the nerve fibers at a higher magnification, regular spaced neurofilaments and microtubules can be identified. Moreover, vesicles with the characteristic size and localization of synaptic vesicles are frequently found within these nerve fibers.

Further quantitative real time PCR (qPCR) analysis demonstrates increasing expression levels of various neural (DCX, Map2, NEFL, NeuN, TBR2, TUBB3, Syt1), midbrain (EN1, GIRK2, MIXL1, NURR1, TH), and glia-specific (GLAST, MBP, S100b) markers at different developmental stages with concomitant decreases in neural precursor markers (Brn2, nestin, Pax6, Sox1, Sox2), confirming neural midbrain maturation over time (FIG. 3).

### Calcium Imaging Reveals Spontaneous and Synchronized Activity Throughout Entire Organoids

To assess the functional coupling of individual cells within the AMOs we performed Fluo-4 acetoxymethyl ester (AM)-based calcium imaging, which can be used as a readout for spiking activity of neurons (Grienberger, C. & Konnerth, A. Imaging calcium in neurons. Neuron 73, 862-885 (2012)). In addition to spontaneous activity of individual cells, we observed organoid-wide synchronous and periodic calcium spikes in all analyzed organoids (n=5). To characterize this behavior further, we defined different regions of interest (ROIs) and assessed the change in fluorescence intensity over time in each region (FIG. 4). Measuring the entire organoid reveals two consecutive spikes in Fluo-4 brightness, with a period of approximately 30 seconds (FIG. 4a). When we subdivide the measured area into 4 quadrants, we see synchronized spiking activity in all 4 resulting ROIs (FIG. 4b). This parallel activity pattern can be found at many structural levels of the organoid, even for single cells (FIG. 4c). Changing the time scale reveals additional levels of synchronicity between selected single cells, in addition to the overall organoid-wide spikes (FIG. 4d). Considered along with the existence of synaptic vesicles on the ultrastructural level (Figure S2) and synapses via immunostaining (FIG. 2h) as well as synaptotagmin 1 (Syt1) via qPCR (FIG. 3), the calcium imaging results support the presence of functionally coupled neurons within the AMOs. The synchronous spiking patterns suggest that not only a few neurons but, in fact, the entire organoid is functionally connected.

### RNA Sequencing Reveals Lower Intra- and Inter-Batch Variability in Automated Midbrain Organoids Compared to Established Protocols

To examine the variance of AMOs on the gene expression level, we performed RNA sequencing of single organoids from three different batches of AMOs and one batch of manually produced iPSC-derived organoids following the established protocol from Lancaster et al. (Cerebral organoids model human brain development and microcephaly. Nature 501, 373-379 (2013)) as controls. We sequenced the AMOs at day 30 and the iPSC-derived organoids at day 30 and 45 for a more equivalent comparison, as the latter need to pass through a neural progenitor phase first before entering their neural maturation. This comparison revealed that AMOs are more reproducible intra- and inter-batch than current standard protocols, as the dispersion of whole genome expression levels measured via principal component analysis (PCA, FIG. 5a) in AMOs is approximately 4 times lower than in the iPSC-derived organoids (FIG. 5b).

Further gene ontology (GO) (Ashburner, M. et al. Gene ontology: tool for the unification of biology. The Gene Ontology Consortium. Nat Genet 25, 25-29 (2000); and Supek, F., Bosnjak, M., Skunca, N. & Smuc, T. REVIGO summarizes and visualizes long lists of gene ontology terms. PLoS One 6, e21800 (2011)) analysis of the genes significantly upregulated (padj.<0.05) in day 30 AMOs compared to day 45 iPSC-derived organoids (FIG. 5c) yielded almost exclusively GO terms connected to neuronal maturation, especially synaptic activity (FIG. 5d, for a complete list of GO terms and further analysis see Table 1) indicating that AMOs mature faster than established organoids.

In screening settings, the wells at the edges of plates often display different readouts than those located further towards the center of the plate (“edge-effects”) (Malo, N., Hanley, J. A., Cerquozzi, S., Pelletier, J. & Nadon, R. Statistical practice in high-throughput screening data analysis. Nat Biotechnol 24, 167-175 (2006)). Therefore, we decided to sequence half of a 96-well plate for one AMO batch and tested for differences resulting from well location within the plate (group “1 inside”=center plate vs. “1 outside”=edge in FIG. 5a/b). The AMOs cluster independently of their position on the plate, indicating that AMOs exhibit no measurable edge effects on the gene expression level. Taken together, the RNA sequencing results illustrate a higher homogeneity as well as faster neuronal maturation of AMOs compared to standard iPSC-based protocols.

### Automated Whole Mount Immunostaining is Highly Quantitative 166 and Reveals Homogeneity of Automated Midbrain Organoids

While immunofluorescence-based screening-compatible techniques of whole organoids have been reported, they can only detect cells in the outer layers of large organoids (Vergara, M. N. et al. Three-dimensional automated reporter quantification (3D-ARQ) technology enables quantitative screening in retinal organoids. Development 144, 3698-3705 (2017)), use small aggregates of approximately 100 μm diameter (Verissimo, C. S. et al. Targeting mutant RAS in patient-derived colorectal cancer organoids by combinatorial drug screening. Elife 5 (2016)), or cystic organoids (Czerniecki, S. M. et al. High-Throughput Screening Enhances Kidney Organoid Differentiation from Human Pluripotent Stem Cells and Enables Automated Multidimensional Phenotyping. Cell Stem Cell 22, 929-940 e924 (2018)) that can be penetrated by antibodies and fluorescence illumination more easily. In contrast, our workflow allows the quantification of entire dense, large-scale organoids (>800 μm diameter) with single cell resolution and high sensitivity, as highlighted by a dose-response assay for 3D cellular detection (FIG. 6a). We mixed cells tracked with a fluorescent dye with unlabeled cells at known proportions, aggregated them, cleared them, and then analyzed them on a confocal high-content imaging system. The resulting relationship between the amount of tracked cells and measured brightness is highly linear (R2>0.99), illustrating the quantitative nature of our optical HTS 3D whole mount analysis workflow.

Next, we demonstrated the homogeneity of AMOs on the protein level. A fully automated 96-well based AMO whole mount optical analysis (FIG. 6b left) illustrates the ability to detect both abundant filamentous structures (neural marker Map2) and nuclear markers (Sox2) (FIG. 6b right, single slice from one organoid) in a HTS-compatible manner. Using nuclear markers like Sox2, our technique allows quantification at single cell resolution by identifying, counting, and summing the brightness of Sox2+ nuclei for each imaged confocal plane (FIG. 6c/d/f/h). Filamentous, abundant signals like Map2 can be quantified throughout organoids by summing the overall mean brightness for each confocal plane (FIG. 6e/g). The comparison of three 96 well plates from independent differentiations revealed the uniform cellular composition of AMOs within and between batches (FIG. 6d-g) (Average CVSox2=5%, CVMap2=9%).

Positional analysis detected effects of plate position (edge effects) for Map2 levels but not Sox2 levels with about 10% reduced Map2 brightness of organoids in the center of the plate (FIG. S3) compared with the wells at the edge. Considered together with the absence of edge effects in the RNA sequencing results, this may indicate that only a specific subset of proteins is altered by edge conditions, while the vast majority of cellular processes is uniform throughout the plate (For a list of differential gene expression between organoids on the inside and edge of the plate see Table 2).

### Automated Midbrain Organoids Allow Toxicity Evaluation in Specific Cellular Subpopulations at the Single Cell Level in a Fully Automated High Throughput Screening Format

To assess the ability of our workflow to quantify drug effects, we treated AMOs with increasing concentrations of the known cytotoxic compound G418 and stained for the apoptosis marker cleaved caspase 3 (cCasp3) together with Sox2. Plotting the number of apoptotic cCasp3+ cells against the logarithmic drug concentration revealed a typical, sigmoidal dose-response curve (FIG. 7a) with an IC50 of 339 μg/mL, which is consistent with published values (Delrue, I., Pan, Q., Baczmanska, A. K., Callens, B. W. & Verdoodt, L. L. M. Determination of the Selection Capacity of Antibiotics for Gene Selection. Biotechnol J 13, e1700747, doi:10.1002/biot.201700747 (2018)). The colocalization analysis between cCasp3 and Sox2 (FIG. 7b) shows that the workflow can also be used to assess cell type specific toxicity in 3D. It also indicates that G418 does not primarily affect Sox2+ neural precursors but other, (more mature) cell types. While the portion of precursors among the apoptotic cells increases with higher inhibitor concentrations, it remains at a low level of only 15% at maximum. FIG. 7c shows examples of the high-content images depicting the increase in cCasp3 signal with increasing inhibitor concentrations. While we stained, imaged, and analyzed entire AMOs for this experiment, we realized during the analysis that considering only a single confocal plane will yield almost identical results. This could be used to considerably reduce the required imaging time and costs in large-scale drug screening campaigns. Finally, we also generated AMOs from a second independent smNPC line derived from patient-derived iPSCs to confirm the applicability of our workflow to different cell lines with a different genetic background.

### Homogeneity—AMOs of the Invention, NABOs of the Invention, and Prior Art Organoids

AMOs are Significantly More Homogeneous than Other Published Brain Organoids with Regard to Overall Morphology and Size

See FIG. 8 and legend.

### The Internal Organization/Structure of State-of-the-Art Brain Organoids is Highly Variable and Unpredictable Compared to AMOs

See FIG. 9 and legend.

### Compared to Only Other Midbrain Organoids, AMOs Still Show the Highest Level of Homogeneity

See FIG. 10 and legend.

### Analysis of Cell Composition Reveals Large Variability in State-of-the-Art Organoids

See FIG. 11 and legend.

### NABOs of the Invention

See FIG. 12 and legend.

1. A method of producing organoids, said method comprising or consisting of:
(a) seeding a plurality of tissue-specific precursor cells into a container;
(b) allowing to occur
(i) aggregation of said cells; and
(ii) maturation of the aggregate formed in (i) into a single organoid;
wherein said method does not comprise embedding of said cells or said aggregates into a gel.

2. The method of claim 1, wherein
(i) said organoids are neural organoids, preferably midbrain organoids or non-patterned homogeneous brain organoids; and
said tissue-specific precursor cells are neuronal tissue-specific precursor cells, preferably small molecule neuronal precursor cells (smNPCs);
(ii) said organoids have a reproducible or homogeneous size and/or cellular composition, homogenous preferably meaning a standard deviation of less than 20% of the mean or less;
(iii) step (b) comprises
(b-i) culturing in aggregation medium, preferably for about two days, said aggregation medium preferably comprising polyvinyl alcohol;
(b-ii) culturing a maturation medium; and
(b-iii) preferably, between (b-i) and (b-ii), culturing in ventral patterning medium, preferably for about four days;
(iv) said plurality of cells is between about 100 and about 1000000, preferably about 10000 cells; and/or
(v) said container is a well of a multiwell plate, wherein preferably a plurality of wells or each well of said multiwell plate is seeded with a plurality of said cells, such that a multiwell plate is obtained, wherein a plurality of wells or each well contains one single organoid.

3. An organoid or a plurality of organoids obtained by the method of any one of the preceding claims.

4. An organoid or a plurality of organoids, wherein
(a) said organoid(s) is/are (a) neural organoid(s), preferably (a) midbrain organoid(s) or (a) non-patterned homogeneous brain organoid(s);
(b) said organoid(s) exhibit(s)
(i) a plurality of concentric zones, each zone differing from any of the other zones with regard to cellular composition and organization, preferably at least three zones; and/or
(ii) said organoid(s) exhibit tissue-specific cellular activity, preferably, in case of neural organoids, electrical activity in neurons; and/or
(c) said plurality of organoids is homogenous in terms of structure and/or size;
wherein said organoid or said plurality is preferably obtained by the method of claim 2.

5. A multiwell plate, wherein a plurality of wells contain each one single organoid or each well contains one single organoid, wherein preferably a plurality of the organoids or each organoid is as defined in claim 3 or 4 or obtained by the method of claim 1 or 2.

6. Use of tissue-specific precursor cells for organoid production, wherein no use is made of a gel for embedding cells or aggregates, wherein preferably said tissue-specific precursor cells are neuronal tissue-specific precursor cells, preferably small molecule neuronal precursor cells (smNPCs).

7. A method of preparing organoids or spheroids for analysis, said method comprising or consisting of:
(a) staining said organoids or spheroids;
(b) performing tissue clearing with said organoids or spheroids.

8. The method of claim 7, wherein
(a) said staining is effected with
(i) an antibody, preferably with a primary and with a secondary antibody, wherein staining with said primary antibody and/or said secondary antibody is effected for about 5 to about 10 days, preferably about 6 days;
(ii) a fluorescent label;
(iii) a luminescent label;
(iv) a radioactive label;
and/or
(b) said clearing is benzyl alcohol and benzyl benzoate (BABB)-based clearing, wherein preferably said clearing is performed in cyclo-olefin containers, more preferably in cyclo-olefin multiwell plates.

9. The method of claim 7 or 8, wherein
(a) said method does not comprise sectioning of said organoids or spheroids and/or said staining is whole mount staining; and/or
(b) said organoids are organoids of claim 3 or 4 or are obtained by the method of claim 1 or 2.

10. A method of analysing organoids or spheroids, said method comprising or consisting of the method of any one of claims 7 to 9; and
(c) analysis of stained and cleared organoids or spheroids, preferably
(c-i) optical analysis, said optical analysis preferably comprising microscopy and/or image analysis;
(c-ii) genetic analysis such as RNA sequencing; and/or
(c-iii) protein analysis such as mass spectrometry or Western blotting.

11. A method of preparing and analysing organoids, said method comprising or consisting of the method of claim 1 or 2 and the method of claim 10.

12. A method of identifying modulators of organoids, of organoid formation, and/or of organoid-specific function, said method comprising or consisting of
(a) (i) adding a test compound to an organoid, preferably of claim 3 or 4 or obtained by the method of claim 1 or 2;
(ii) adding a test compound to tissue-specific precursor cells, followed by performing the method of claim 1 or 2; or
(iii) performing the method of claim 1 or 2, wherein a test compound is added at one or more time points during said performing the method of claim 1 or 2;
(b) performing the method of claim 10;
(c) comparing the result of said analysis in the presence of said test compound with the result of said analysis in the absence of said test compound, wherein a difference is indicative of a modulator.

13. The method of claim 12, wherein
(a) if said analysis is indicative of a functional improvement of said organoid, of organoid formation and/or of organoid-specific function, said test compound is a lead compound, said method optionally further comprising or further consisting of developing said lead compound to yield a drug; or
(b) if said analysis is indicative of a decrease of function of said organoid and/or of negative interference with organoid formation and/or with organoid-specific function, this is indicative of said test compound being toxic.

14. The method of any one of claims 1, 2 or 7 to 13, wherein said method is performed
(a) in an automated manner; and/or
(b) in high-throughput format, preferably using multiwell plates, a pipetting robot, automated liquid handling, a plate reader and/or means for plate transportation.

15. A kit comprising or consisting of
(a) tissue-specific precursor cells, preferably neuronal tissue-specific precursor cells, more preferably smNPCs; and
(b) media, said media comprising or consisting of
(b-i) aggregation medium, said aggregation medium preferably comprising polyvinyl alcohol;
(b-ii) maturation medium; and
(b-iii) optionally, ventral patterning medium.
